SEQUENCE LISTING (45 sequences)

SEQ ID NO: 1 - protein, 44 aa, synthetic construct
  REGION 1..44: note = Synthetic Polypeptide
KYYGNGVTCGKHSCSVDWGKATTCIINNGAMAWATGGHQGNHKC

SEQ ID NO: 2 - protein, 44 aa, synthetic construct; identical to SEQ ID NO: 1
  REGION 1..44: note = Synthetic Polypeptide
  DISULFID 9..14: note = Intrachain disulfide bond
  DISULFID 24..44: note = Intrachain disulfide bond
  SITE 31: note = MISC_FEATURE - Methionine sulfoxide

SEQ ID NO: 3 - protein, 44 aa, synthetic construct; identical to SEQ ID NO: 1
  REGION 1..44: note = Synthetic Polypeptide
  DISULFID 9..24: note = Intrachain disulfide bond
  DISULFID 14..44: note = Intrachain disulfide bond

SEQ ID NO: 4 - protein, 44 aa, synthetic construct; identical to SEQ ID NO: 1
  REGION 1..44: note = Synthetic Polypeptide
  DISULFID 9..44: note = Intrachain disulfide bond
  DISULFID 14..24: note = Intrachain disulfide bond

SEQ ID NO: 5 - protein, 44 aa, synthetic construct; identical to SEQ ID NO: 1
  REGION 1..44: note = Synthetic Polypeptide
  DISULFID 9..14: note = Intrachain disulfide bond
  DISULFID 24..44: note = Intrachain disulfide bond

SEQ ID NO: 6 - protein, 44 aa, synthetic construct
  REGION 1..44: note = Synthetic Polypeptide
KYYGNGVTCGKHSCSVDWGKATTCIINNGALAWATGGHQGNHKC

SEQ ID NO: 7 - protein, 44 aa, synthetic construct; identical to SEQ ID NO: 6
  REGION 1..44: note = Synthetic Polypeptide
  DISULFID 9..14: note = Intrachain disulfide bond
  DISULFID 24..44: note = Intrachain disulfide bond

SEQ ID NO: 8 - protein, 44 aa, synthetic construct
  REGION 1..44: note = Synthetic Polypeptide
KYYGNGVTAGKHSASVDWGKATTAIINNGAMAWATGGHQGNHKA

SEQ ID NO: 9 - protein, 22 aa, synthetic construct
  REGION 1..22: note = Synthetic Polypeptide
KRKKHRCRVYNNGMPTGMYRWC

SEQ ID NO: 10 - protein, 22 aa, synthetic construct
  REGION 1..22: note = Synthetic Polypeptide
KRKKHRCRVYNNGLPTGLYRWC

SEQ ID NO: 11 - protein, 22 aa, synthetic construct
  REGION 1..22: note = Synthetic Polypeptide
KRKKHRCRVYNNGMPTGMYRWC

SEQ ID NO: 12 - protein, 22 aa, synthetic construct
  REGION 1..22: note = Synthetic Polypeptide
KRKKHRCRVYNNGLPTGLYRWC

SEQ ID NO: 13 - protein, 22 aa, synthetic construct
  REGION 1..22: note = Synthetic Polypeptide
  MOD_RES 22: note = AMIDATION
KRKKHRCRVYNNGLPTGLYRWC

SEQ ID NO: 14 - protein, 22 aa, synthetic construct
  REGION 1..22: note = Synthetic Polypeptide
  MOD_RES 1: note = ACETYLATION
  MOD_RES 22: note = AMIDATION
KRKKHRCRVYNNGLPTGLYRWC

SEQ ID NO: 15 - protein, 19 aa, synthetic construct
  REGION 1..19: note = Synthetic Polypeptide
  MOD_RES 19: note = AMIDATION
KHRCRVYNNGLPTGLYRWC

SEQ ID NO: 16 - protein, 18 aa, synthetic construct
  REGION 1..18: note = Synthetic Polypeptide
  MOD_RES 18: note = AMIDATION
HRCRVYNNGLPTGLYRWC

SEQ ID NO: 17 - protein, 17 aa, synthetic construct
  REGION 1..17: note = Synthetic Polypeptide
  MOD_RES 17: note = AMIDATION
RCRVYNNGLPTGLYRWC

SEQ ID NO: 18 - protein, 16 aa, synthetic construct
  REGION 1..16: note = Synthetic Polypeptide
  MOD_RES 16: note = AMIDATION
CRVYNNGLPTGLYRWC

SEQ ID NO: 19 - protein, 22 aa, synthetic construct
  REGION 1..22: note = Synthetic Polypeptide
  MOD_RES 22: note = AMIDATION
KRKKHRCRVFNNGLPTGLYRWC

SEQ ID NO: 20 - protein, 22 aa, synthetic construct
  REGION 1..22: note = Synthetic Polypeptide
  MOD_RES 22: note = AMIDATION
KRKKHRCRVWNNGLPTGLYRWC

SEQ ID NO: 21 - protein, 22 aa, synthetic construct
  REGION 1..22: note = Synthetic Polypeptide
  MOD_RES 22: note = AMIDATION
KRKKHRCRVYNNGLPTGLFRWC

SEQ ID NO: 22 - protein, 22 aa, synthetic construct
  REGION 1..22: note = Synthetic Polypeptide
  MOD_RES 22: note = AMIDATION
KRKKHRCRVYNNGLPTGLSRWC

SEQ ID NO: 23 - protein, 22 aa, synthetic construct
  REGION 1..22: note = Synthetic Polypeptide
  MOD_RES 22: note = AMIDATION
KRKKHRCRVYNNGLPTGLWRWC

SEQ ID NO: 24 - protein, 22 aa, synthetic construct
  REGION 1..22: note = Synthetic Polypeptide
  MOD_RES 22: note = AMIDATION
ARKKHRCRVYNNGLPTGLYRWC

SEQ ID NO: 25 - protein, 22 aa, synthetic construct
  REGION 1..22: note = Synthetic Polypeptide
  MOD_RES 22: note = AMIDATION
KAKKHRCRVYNNGLPTGLYRWC

SEQ ID NO: 26 - protein, 22 aa, synthetic construct
  REGION 1..22: note = Synthetic Polypeptide
  MOD_RES 22: note = AMIDATION
KRAKHRCRVYNNGLPTGLYRWC

SEQ ID NO: 27 - protein, 22 aa, synthetic construct
  REGION 1..22: note = Synthetic Polypeptide
  MOD_RES 22: note = AMIDATION
KRKAHRCRVYNNGLPTGLYRWC

SEQ ID NO: 28 - protein, 22 aa, synthetic construct
  REGION 1..22: note = Synthetic Polypeptide
  MOD_RES 22: note = AMIDATION
KRKKARCRVYNNGLPTGLYRWC

SEQ ID NO: 29 - protein, 22 aa, synthetic construct
  REGION 1..22: note = Synthetic Polypeptide
  MOD_RES 22: note = AMIDATION
KRKKHACRVYNNGLPTGLYRWC

SEQ ID NO: 30 - protein, 22 aa, synthetic construct
  REGION 1..22: note = Synthetic Polypeptide
  MOD_RES 22: note = AMIDATION
KRKKHRARVYNNGLPTGLYRWC

SEQ ID NO: 31 - protein, 22 aa, synthetic construct
  REGION 1..22: note = Synthetic Polypeptide
  MOD_RES 22: note = AMIDATION
KRKKHRCAVYNNGLPTGLYRWC

SEQ ID NO: 32 - protein, 22 aa, synthetic construct
  REGION 1..22: note = Synthetic Polypeptide
  MOD_RES 22: note = AMIDATION
KRKKHRCRAYNNGLPTGLYRWC

SEQ ID NO: 33 - protein, 22 aa, synthetic construct
  REGION 1..22: note = Synthetic Polypeptide
  MOD_RES 22: note = AMIDATION
KRKKHRCRVANNGLPTGLYRWC

SEQ ID NO: 34 - protein, 22 aa, synthetic construct
  REGION 1..22: note = Synthetic Polypeptide
  MOD_RES 22: note = AMIDATION
KRKKHRCRVYANGLPTGLYRWC

SEQ ID NO: 35 - protein, 22 aa, synthetic construct
  REGION 1..22: note = Synthetic Polypeptide
  MOD_RES 22: note = AMIDATION
KRKKHRCRVYNAGLPTGLYRWC

SEQ ID NO: 36 - protein, 22 aa, synthetic construct
  REGION 1..22: note = Synthetic Polypeptide
  MOD_RES 22: note = AMIDATION
KRKKHRCRVYNNALPTGLYRWC

SEQ ID NO: 37 - protein, 22 aa, synthetic construct
  REGION 1..22: note = Synthetic Polypeptide
  MOD_RES 22: note = AMIDATION
KRKKHRCRVYNNGAPTGLYRWC

SEQ ID NO: 38 - protein, 22 aa, synthetic construct
  REGION 1..22: note = Synthetic Polypeptide
  MOD_RES 22: note = AMIDATION
KRKKHRCRVYNNGLATGLYRWC

SEQ ID NO: 39 - protein, 22 aa, synthetic construct
  REGION 1..22: note = Synthetic Polypeptide
  MOD_RES 22: note = AMIDATION
KRKKHRCRVYNNGLPAGLYRWC

SEQ ID NO: 40 - protein, 22 aa, synthetic construct
  REGION 1..22: note = Synthetic Polypeptide
  MOD_RES 22: note = AMIDATION
KRKKHRCRVYNNGLPTALYRWC

SEQ ID NO: 41 - protein, 22 aa, synthetic construct
  REGION 1..22: note = Synthetic Polypeptide
  MOD_RES 22: note = AMIDATION
KRKKHRCRVYNNGLPTGAYRWC

SEQ ID NO: 42 - protein, 22 aa, synthetic construct
  REGION 1..22: note = Synthetic Polypeptide
  MOD_RES 22: note = AMIDATION
KRKKHRCRVYNNGLPTGLARWC

SEQ ID NO: 43 - protein, 22 aa, synthetic construct
  REGION 1..22: note = Synthetic Polypeptide
  MOD_RES 22: note = AMIDATION
KRKKHRCRVYNNGLPTGLYAWC

SEQ ID NO: 44 - protein, 22 aa, synthetic construct
  REGION 1..22: note = Synthetic Polypeptide
  MOD_RES 22: note = AMIDATION
KRKKHRCRVYNNGLPTGLYRAC

SEQ ID NO: 45 - protein, 22 aa, synthetic construct
  REGION 1..22: note = Synthetic Polypeptide
  MOD_RES 22: note = AMIDATION
KRKKHRCRVYNNGLPTGLYRWA

(19) United States
(12) Patent Application Publication
(10) Pub. No.: US 2023/0337702 A1
(43) Pub. Date: Oct. 26, 2023

(54) PROCESS FOR THE LINEAR SYNTHESIS OF GRAM-POSITIVE CLASS II BACTERIOCINS AND COMPOSITIONS AND USES THEREOF

(71) Applicant: UNIVERSITÉ LAVAL, Québec (CA)

(72) Inventors: François Bédard, Saint-Augustin-De-Desmaures (CA); Éric Biron, Saint-Augustin-De-Desmaures (CA); Riadh Hammami, Ottawa (CA); Ismail Fliss, Québec (CA); Ariane RENAUD, Québec (CA); Laurent DALLAIRE, Québec (CA)

(21) Appl. No.: 18/087,433

(22) Filed: Dec. 22, 2022

Related U.S. Application Data
(63) Continuation-in-part of application No. 16/616,322, filed on Nov. 22, 2019, now Pat. No. 11,571,456.
(60) Provisional application No. 62/509,978, filed on May 23, 2017.

(51) Int. Cl. A23L 3/3463; C07K 14/32

(52) U.S. Cl. CPC A23L 3/34635; C07K 14/32

(57) ABSTRACT

A process for the linear synthesis of a gram-positive class II bacteriocin or a variant thereof is disclosed herein. The process comprises the stepwise addition of selected amino acids to a solid support; pseudoproline positioning and reopening; and cleavage of the gram-positive class II bacteriocin or the variant thereof from the solid support to provide a linear gram-positive class II bacteriocin or variant thereof; and in situ disulfide bond formation. Various applications and uses of the synthetic bacteriocins are also disclosed. The synthetic process can also be used to synthesize variants of bacteriocins by the selective substitution of one or more amino acids and/or additions and/or deletions of selected amino acids.

### CROSS REFERENCE TO RELATED APPLICATIONS

This application is a continuation-in-part of U.S. Pat. Application 16/616,322 filed Nov. 22, 2019, which is a national phase under 35 U.S.C. § 371 of International Application No. PCT/CA2018/050598 filed May 23, 2018, which claims the benefit of U.S. Provisional Application 62/509,978 filed May 23, 2017. The contents of each application are hereby incorporated into the present application by reference.

### SEQUENCE LISTING

This application contains a Sequence Listing which has been submitted via EFS-Web and is hereby incorporated by reference in its entirety. Said copy, created on Oct. 12, 2022 is named “LVRYP0007US_ST25” and is 17.8 kb in size.

### FIELD

The present disclosure broadly relates to a process for the linear synthesis of gram-positive class II bacteriocins, variants thereof, and compositions and uses thereof. More specifically, but not exclusively, the present disclosure broadly relates to a process for the linear synthesis of gram-positive class IIa, IIb, IIc and IId bacteriocins, variants thereof, and compositions and uses thereof. Yet more specifically, but not exclusively, the present disclosure relates to a process for the linear synthesis of pediocin PA-1, variants thereof, and compositions and uses thereof. Yet more specifically, but not exclusively, the present disclosure relates to a process for the linear synthesis of bactofencin A, variants thereof, and compositions and uses thereof. The present disclosure also relates to novel antimicrobial agents.

### BACKGROUND

Antibiotic therapy is certainly one of the most important scientific achievements of the twentieth century both in terms of economic and health impacts for animals as well as for humans. However, the abuse of antibiotics, such as by overprescription, has led to an alarming increase in the number of multi-resistant pathogenic strains such as MRSA (Methicillin Resistant Staphylococcus aureus), VRE (Vancomycin Resistant Enterococci) and Clostridium difficile and the appearance of serious health problems including significant damage to human commensal microflora and certain autoimmune diseases.[1]

The increase in resistance to antibiotics has become a major challenge in the treatment of various bacterial infections. Indeed, the use of large-scale antibiotics has accelerated the emergence of resistance in many bacterial species. In the US alone, according to recent reports by the Centers for Disease Control and Prevention (CDC), there are at least 2 million people suffering from bacterial related infections, and 23000 deaths per year have been reported that are linked to antibiotic-resistant bacteria. In view of the first generations of antibiotics losing their effectiveness, as evidenced by the aforementioned resistance, a substantial amount of research is being dedicated to the discovery and development of novel and alternative antibiotics.

Bacteriocins represent a class of antimicrobial peptides (AMPs) produced by a broad variety of bacteria in order to survive in their respective competitive environments. Potential health risks associated with the use of chemical preservatives, in addition to increased consumer awareness, have led to bacteriocins being given greater consideration as natural antimicrobial agents.[2] The substantial absence of cross-resistance, as well as the faint propensity for the development of resistance, are further factors favoring the use of bacteriocins as antibiotic agents.[3, 4] Moreover, several combinations of bacteriocins and antibiotics have been reported as exhibiting synergistic effects.[5] Furthermore, bacteriocins have been considered for use in both medicine and the food industry. To date, only one bacteriocin (i.e., nisin A; commercialised as Nisaplin®) has been approved by the Food and Drug Administration (FDA) as “Generally Recognized As Safe” (GRAS). The market value of nisin is expected to reach 500 M$ in 2020.

Among the bacteriocins, the class IIa bacteriocins are produced from food grade bacteria, which offer food scientists the possibility of directing or preventing the development of specific bacterial species in food. This can be particularly useful in preservation or food safety applications. Indeed, the class IIa bacteriocins are active against pathogens such as Listeria monocytogenes which has a mortality rate as high as 20-30% when infected. Moreover, Listeria monocytogenes has been associated with neonatal deaths, fetal demise, severe meningitis and sepsis.

Even though a great many bacteriocins have been sequenced, only very few have actually been chemically synthesized. Indeed, their synthesis represents, and continuous to represent, a significant challenge to the peptide chemist, largely due to their inherent structural complexity and well defined structure activity relationships (SAR).[6] Moreover, purified bacteriocins isolated from fermentation batches remain quite expensive, partly due to a lengthy purification process, prohibiting their commercial-scale exploitation. [7] The development of chemical syntheses provides for the added advantage of enhancing the pharmacological properties of a given bacteriocin (e.g. solubility, stability, potency, activity and bioavailability). Among the class IIa bacteriocins, only a very few called “pediocin-like” bacteriocins, have actually been successfully synthesized. Similarly, very few class IIb-e bacteriocins have been successfully synthesized. To that effect, the total synthesis of Pediocin PA-1 and bactofencin A has been previously achieved in extremely low yields (~ 0.1% overall yield) and only a few analogs have been prepared. Pediocin PA-1 (class IIa bacteriocin) is a broad-spectrum lactic acid bacteria bacteriocin that shows a particularly strong activity against Listeria monocytogenes. Bactofencin A (class IId bacteriocin) shows strong activity against pathogens Listeria monocytogenes and Staphylococcus aureus.

The relevance of developing a processing aid against a microbiological target such as Listeria monocytogenes would be advantageous. This microorganism has the highest mortality rate amongst foodborne bacteria in North America. [8] According to the Canadian Food Inspection Agency (CFIA) between 2014 and March 2019, 214 recalls were attributed to contamination by Listeria monocytogenes in various food categories (Table 1). In the US, between 2017 and 2022, 269 recalls have been listed by the FDA for Listeria only. [9] These numbers are quite impressive considering the huge repercussion of recalls for the food industry. The costs of a recall for a company are estimated to be about $10M.[10] These costs do not take into account that 55% of consumers will change brands and this will result in further loss of revenue for the company. Moreover, each year, 4M Canadians are affected by foodborne diseases . According to the CDC, of this 4M, 1600 people contract listeriosis and 260 have died of listeriosis.[11] The mortality rate of listeriosis is 15-50%. This foodborne disease affects a wide spectrum of people, from immunosuppressed persons, pregnant women, and the elderly (over 60 years old).

TABLE 1

Number of microbiological recalls caused by Listeria monocytogenes or Clostridium Botulinum in Canada between 2014 and March 2019 (CFIA)

| Food categories | Number of recalls / contaminants | | Total number of recalls |
|---|---|---|---|
| | Listeria monocytogenes | Clostridium botulinum | |
| Lettuce and ready-to eat food | 71 | 16 | 87 |
| Fruits and vegetables | 57 | - | 57 |
| Cheese | 29 | - | 29 |
| Fishes and Seafood | 12 | 41 | 53 |
| Meat and deli | 19 | 3 | 22 |
| Milk products | 11 | - | 11 |
| Poultry | 10 | - | 10 |

The effectiveness of pediocin PA-1 against Listeria monocytogenes, and more recently of pediocin PA-1 M31L, is well documented. [12-15] The latter has also been shown to be effective against certain strains of Gram-positive bacteria such as Carnobacterium divergens, Leuconostoc mesenteroides, Clostridium perfringens and Lactobacillus plantarum (Table 2).[15-18] The minimal inhibitory concentration (MIC) of pediocin PA-1 M31L against these pathogens is very low, compared to the MIC of antibiotics for the same strains. For Listeria monocytogenes, pediocin PA-1 M31L’s MIC is 13 nM (60 ppb), relatively to penicillin’s MIC of 373.8 - 1495 nM (0.125-0.5 ppm) in tryptic soy broth (TSB) medium.[19] Listeria monocytogenes is up to 1000 times more sensitive to pediocin PA-1 M31L than a common antibiotic. Pediocin PA-1 M31L, and bacteriocins in general, represent a potential solution in response to the multi-drug resistance pathogens. [20] The increasing rate of antibiotic resistance is one of the main driving forces for food processors to eliminate the use of antibiotics throughout the food processing chain.

TABLE 2

Pediocin PA-1 M31L spectrum of activity against various bacteria evaluated by the determination of the minimal inhibitory concentration (MIC)

| Strains | Reference | IC (nM) | Diffusion (mm) | Culture media |
|---|---|---|---|---|
| Carnobacterium divergens | ATCC 35677 | 1.9 | 35 | BHI |
| Leuconostoc mesenteroides | ATCC 23386 | 1.9 | 33 | MRS |
| Listeria seeligeri | ATCC 35967 | 4.7 | 32 | TSBYE |
| Listeria monocytogenes | ATCC 19111 | 13.5 | 31 | TSBYE |
| Clostridium perfringens | ATCC 1-222 | 37.8 | 25 | RCM |
| Clostridium perfringens | ATCC 1-223 | 75.7 | 22 | RCM |
| Listeria murrayi | ATCC 25401 | 151.4 | 28 | TSBYE |
| Lactobacillus plantarum | ATCC 8014 | 605.5 | 23 | MRS |

The present disclosure refers to a number of documents, the contents of which are herein incorporated by reference in their entirety.

### SUMMARY

In an aspect, the present disclosure broadly relates to a process for the linear synthesis of gram-positive class II bacteriocins, variants thereof, and compositions and uses thereof.

In an aspect, the present disclosure broadly relates to novel antibacterial peptides. More specifically, but not exclusively, the present disclosure relates to novel gram-positive class II bacteriocins and variants thereof. Yet more specifically, but not exclusively, the present disclosure relates to a process for the linear synthesis of gram-positive class II bacteriocins, variants thereof and compositions and uses thereof. The present disclosure also relates to a process for the linear synthesis of gram-positive class II bacteriocins comprising a disulfide bond, variants thereof and compositions and uses thereof. The present disclosure also relates to a process for the linear synthesis of pediocin PA-1, variants thereof and compositions and uses thereof. The present disclosure also relates to a process for the linear synthesis of bactofencin A, variants thereof and compositions and uses thereof. In an embodiment of the present disclosure, the process for the linear synthesis of gram-positive class II bacteriocins comprises the use of solid phase peptide synthesis. In a further embodiment of the present disclosure, the process for the linear synthesis of gram-positive class II bacteriocins comprising a disulfide bond includes the use of solid phase peptide synthesis. In a further embodiment of the present disclosure, the process for the linear synthesis of pediocin PA-1 and variants thereof comprises the use of solid phase peptide synthesis. In a further embodiment of the present disclosure, the process for the linear synthesis of bactofencin A and variants thereof comprises the use of solid phase peptide synthesis.

In an aspect, the present disclosure relates to a process for the linear synthesis of a bacteriocin, the process comprising the stepwise addition of selected amino acids to a solid support; pseudoproline positioning and reopening; and cleavage of the bacteriocin from the solid support to provide a linear bacteriocin. In an embodiment of the present disclosure, the process further comprises in situ formation of a disulfide bond. In an embodiment of the present disclosure the bacteriocin is at least one of pediocin PA-1 or bactofencin A.

In an aspect, the present disclosure relates to a process for the linear synthesis of a bacteriocin or a variant thereof, the process comprising the stepwise addition of selected amino acids to a solid support; pseudoproline positioning and reopening; and cleavage of the bacteriocin or variant thereof from the solid support to provide a linear bacteriocin or variant thereof. In an embodiment of the present disclosure, the process further comprises in situ formation of a disulfide bond. In an embodiment of the present disclosure the bacteriocin is at least one of pediocin PA-1 or bactofencin A or variants thereof.

In an aspect, the present disclosure relates to a process for the linear synthesis of a bacteriocin, the process comprising the stepwise addition of selected amino acids to a solid support; pseudoproline positioning and reopening; cleavage of the bacteriocin from the solid support to provide a linear bacteriocin; and disulfide bond formation. In an embodiment of the present disclosure, the disulfide formation occurs in situ. In a further embodiment of the present disclosure the bacteriocin is at least one of pediocin PA-1 or bactofencin A.

In an aspect, the present disclosure relates to a process for the linear synthesis of a bacteriocin or a variant thereof, the process comprising the stepwise addition of selected amino acids to a solid support; pseudoproline positioning and reopening; cleavage of the bacteriocin or variant thereof from the solid support to provide a linear bacteriocin or variant thereof; and disulfide bond formation. In an embodiment of the present disclosure, the disulfide formation occurs in situ. In a further embodiment of the present disclosure the bacteriocin is at least one of pediocin PA-1 or bactofencin A or variants thereof.

In an aspect, the present disclosure relates to a process for the linear synthesis of a bacteriocin, the process comprising the stepwise addition of selected amino acids to a solid support; pseudoproline positioning and reopening; cleavage of the bacteriocin from the solid support to provide a linear bacteriocin; and disulfide bond formation. In an embodiment of the present disclosure, the disulfide formation occurs in situ. In a further embodiment of the present disclosure, the process further comprises treating the linear bacteriocin with a mobile phase comprising an acid. In an embodiment of the present disclosure the bacteriocin is at least one of pediocin PA-1 or bactofencin A.

In an aspect, the present disclosure relates to a process for the linear synthesis of a bacteriocin or a variant thereof, the process comprising the stepwise addition of selected amino acids to a solid support; pseudoproline positioning and reopening; cleavage of the bacteriocin or variant thereof from the solid support to provide a linear bacteriocin or variant thereof; and disulfide bond formation. In an embodiment of the present disclosure, the disulfide formation occurs in situ. In a further embodiment of the present disclosure, the process further comprises treating the linear bacteriocin or variant thereof with a mobile phase comprising an acid. In an embodiment of the present disclosure the bacteriocin is at least one of pediocin PA-1 or bactofencin A or variants thereof.

In an aspect, the present disclosure relates to a process for the linear synthesis of a bacteriocin, the process comprising the stepwise addition of selected amino acids to a solid support; pseudoproline positioning and reopening; cleavage of the bacteriocin from the solid support to provide a linear bacteriocin; and disulfide bond formation. In a further embodiment of the present disclosure, the process further comprises treating the linear bacteriocin with a mobile phase comprising an acid. In a further embodiment of the present disclosure, the disulfide bond formation comprises oxidative coupling of a pair of thiol containing amino acid residues using an oxidant. In a further embodiment of the present disclosure, the disulfide bond formation comprises in situ disulfide bond formation by contacting the bacteriocin with a suitable medium, non-limiting examples of which include bioassays, biological media or a food matrix. In an embodiment of the present disclosure the bacteriocin is at least one of pediocin PA-1 or bactofencin A.

In an aspect, the present disclosure relates to a process for the linear synthesis of a bacteriocin or a variant thereof, the process comprising the stepwise addition of selected amino acids to a solid support; pseudoproline positioning and reopening; cleavage of the bacteriocin or a variant thereof from the solid support to provide a linear bacteriocin or a variant thereof; and disulfide bond formation. In a further embodiment of the present disclosure, the process further comprises treating the linear bacteriocin or a variant thereof with a mobile phase comprising an acid. In a further embodiment of the present disclosure, the disulfide bond formation comprises oxidative coupling of a pair of thiol containing amino acid residues using an oxidant. In a further embodiment of the present disclosure, the disulfide bond formation comprises in situ disulfide bond formation by contacting the bacteriocin or a variant thereof with a suitable medium, non-limiting examples of which include bioassays or biological media. In an embodiment of the present disclosure the bacteriocin is at least one of pediocin PA-1 or bactofencin A or variants thereof.

In an aspect, the present disclosure relates to a process for the linear synthesis of a bacteriocin, the process comprising the stepwise addition of selected amino acids to a solid support; pseudoproline positioning and reopening; cleavage of the bacteriocin from the solid support to provide a linear bacteriocin; and in situ disulfide bond formation. In an embodiment of the present disclosure, the process further comprises treating the linear bacteriocin with a mobile phase comprising an acid. In an embodiment of the present disclosure the bacteriocin is at least one of pediocin PA-1 or bactofencin A.

In an aspect, the present disclosure relates to a process for the linear synthesis of a bacteriocin or variants thereof, the process comprising the stepwise addition of selected amino acids to a solid support; pseudoproline positioning and reopening; cleavage of the bacteriocin or variants thereof from the solid support to provide a linear bacteriocin or variants thereof; and in situ disulfide bond formation. In an embodiment of the present disclosure, the process further comprises treating the linear bacteriocin or variants thereof with a mobile phase comprising an acid. In an embodiment of the present disclosure the bacteriocin is at least one of pediocin PA-1 or bactofencin A or variants thereof.

In an aspect, the present disclosure relates to a process for the linear synthesis of a bacteriocin, the process comprising the stepwise addition of selected amino acids to a solid support; pseudoproline positioning and reopening; cleavage of the bacteriocin from the solid support to provide a linear bacteriocin; and in situ disulfide bond formation. In an embodiment of the present disclosure, the process further comprises treating the linear bacteriocin with a mobile phase comprising an acid. In a further embodiment of the present disclosure, the disulfide bond formation comprises in situ disulfide bond formation by contacting the bacteriocin with a suitable medium, non-limiting examples of which include bioassays or biological media. In an embodiment of the present disclosure the bacteriocin is at least one of pediocin PA-1 or bactofencin A.

In an aspect, the present disclosure relates to a process for the linear synthesis of a bacteriocin or variants thereof, the process comprising the stepwise addition of selected amino acids to a solid support; pseudoproline positioning and reopening; cleavage of the bacteriocin or variants thereof from the solid support to provide a linear bacteriocin or variants thereof; and in situ disulfide bond formation. In an embodiment of the present disclosure, the process further comprises treating the linear bacteriocin or variants thereof with a mobile phase comprising an acid. In a further embodiment of the present disclosure, the disulfide bond formation comprises in situ disulfide bond formation by contacting the bacteriocin or variants thereof with a suitable medium, non-limiting examples of which include bioassays or biological media. In an embodiment of the present disclosure the bacteriocin is at least one of pediocin PA-1 or bactofencin A or variants thereof.

In an aspect, the present disclosure relates to a composition comprising a bacteriocin and a pharmacologically acceptable carrier. In an embodiment of the present disclosure, the bacteriocin is a synthetic bacteriocin. In a further embodiment of the present disclosure, the synthetic bacteriocin is obtained by linear solid phase peptide synthesis. In an embodiment of the present disclosure the bacteriocin is at least one of pediocin PA-1 or bactofencin A.

In an aspect, the present disclosure relates to a composition comprising a bacteriocin or a variant thereof, and a pharmacologically acceptable carrier. In an embodiment of the present disclosure, the bacteriocin or variant thereof is a synthetic bacteriocin. In a further embodiment of the present disclosure, the synthetic bacteriocin or variant thereof is obtained by linear solid phase peptide synthesis. In an embodiment of the present disclosure the bacteriocin is at least one of pediocin PA-1 or bactofencin A or variants thereof.

In an aspect, the present disclosure relates to a method for the prevention and/or treatment of bacterial related infections comprising administering to a subject in need thereof an effective amount of a bacteriocin. In an embodiment of the present disclosure, the bacteriocin is a synthetic bacteriocin. In a further embodiment of the present disclosure, the synthetic bacteriocin is obtained by linear solid phase peptide synthesis. In a further embodiment of the present disclosure, the synthetic bacteriocin comprises a disulfide bond. In an embodiment of the present disclosure the bacteriocin is at least one of pediocin PA-1 or bactofencin A.

In an aspect, the present disclosure relates to a method for the prevention and/or treatment of bacterial related infections comprising administering to a subject in need thereof an effective amount of a bacteriocin or a variant thereof. In an embodiment of the present disclosure, the bacteriocin is a synthetic bacteriocin or variant thereof. In a further embodiment of the present disclosure, the synthetic bacteriocin or a variant thereof is obtained by linear solid phase peptide synthesis. In a further embodiment of the present disclosure, the synthetic bacteriocin or a variant thereof comprises a disulfide bond. In an embodiment of the present disclosure the bacteriocin is at least one of pediocin PA-1 or bactofencin A or a variant thereof.

In an aspect, the present disclosure relates to a method of preserving a food item comprising applying an effective amount of a bacteriocin to the food item. In an embodiment of the present disclosure, the bacteriocin is a synthetic bacteriocin. In a further embodiment of the present disclosure, the synthetic bacteriocin is obtained by linear solid phase peptide synthesis. In a further embodiment of the present disclosure, the synthetic bacteriocin comprises a disulfide bond. In a further embodiment of the present disclosure, the synthetic bacteriocin is applied to the surface of the food item. In a further embodiment of the present disclosure, the synthetic bacteriocin is applied within the food item. In an embodiment of the present disclosure the bacteriocin is at least one of pediocin PA-1 or bactofencin A.

In an aspect, the present disclosure relates to a method of preserving a food item comprising applying an effective amount of a bacteriocin or a variant thereof to the food item. In an embodiment of the present disclosure, the bacteriocin is a synthetic bacteriocin or a variant thereof. In a further embodiment of the present disclosure, the synthetic bacteriocin or a variant thereof is obtained by linear solid phase peptide synthesis. In a further embodiment of the present disclosure, the synthetic bacteriocin or a variant thereof comprises a disulfide bond. In a further embodiment of the present disclosure, the synthetic bacteriocin or a variant thereof is applied to the surface of the food item. In an embodiment of the present disclosure the bacteriocin is at least one of pediocin PA-1 or bactofencin A or a variant thereof.

In an aspect, the present disclosure relates to a bactericidal food preservation composition adapted for coating a food product for preservation thereof, the composition comprising a bacteriocin in an amount effective to kill a pathogenic agent upon contact. In an embodiment of the present disclosure, the bacteriocin is a synthetic bacteriocin. In a further embodiment of the present disclosure, the synthetic bacteriocin is obtained by linear solid phase peptide synthesis. In a further embodiment of the present disclosure, the synthetic bacteriocin comprises a disulfide bond. In an embodiment of the present disclosure the bacteriocin is at least one of pediocin PA-1 or bactofencin A.

In an aspect, the present disclosure relates to a bactericidal food preservation composition adapted for coating a food product for preservation thereof, the composition comprising a bacteriocin or a variant thereof in an amount effective to kill a pathogenic agent upon contact. In an embodiment of the present disclosure, the bacteriocin is a synthetic bacteriocin or a variant thereof. In a further embodiment of the present disclosure, the synthetic bacteriocin or a variant thereof is obtained by linear solid phase peptide synthesis. In a further embodiment of the present disclosure, the synthetic bacteriocin or a variant thereof comprises a disulfide bond. In an embodiment of the present disclosure the bacteriocin is at least one of pediocin PA-1 or bactofencin A or a variant thereof.

In an aspect, the present disclosure relates to an item comprising a surface, wherein the surface comprises an antimicrobial effective amount of a bacteriocin. In an embodiment of the present disclosure, the bacteriocin is a synthetic bacteriocin. In a further embodiment of the present disclosure, the synthetic bacteriocin is obtained by linear solid phase peptide synthesis. In a further embodiment of the present disclosure, the synthetic bacteriocin comprises a disulfide bond. In an embodiment of the present disclosure the bacteriocin is at least one of pediocin PA-1 or bactofencin A. In a further embodiment of the present disclosure, the item is selected from the group consisting of a medical device, medical instrument and medical implement.

In an aspect, the present disclosure relates to an item comprising a surface, wherein the surface comprises an antimicrobial effective amount of a bacteriocin or a variant thereof. In an embodiment of the present disclosure, the bacteriocin is a synthetic bacteriocin or a variant thereof. In a further embodiment of the present disclosure, the synthetic bacteriocin or a variant thereof is obtained by linear solid phase peptide synthesis. In a further embodiment of the present disclosure, the synthetic bacteriocin or a variant thereof comprises a disulfide bond. In an embodiment of the present disclosure the bacteriocin is at least one of pediocin PA-1 or bactofencin A or a variant thereof. In a further embodiment of the present disclosure, the item is selected from the group consisting of a medical device, medical instrument and medical implement.

In an aspect, the present disclosure relates to a kit comprising one or more bacteriocins and one or more applicators. In an embodiment of the present disclosure, the one or more bacteriocins are synthetic bacteriocins. In a further embodiment of the present disclosure, the synthetic bacteriocins are obtained by linear solid phase peptide synthesis. In a further embodiment of the present disclosure, the synthetic bacteriocins comprise a disulfide bond. In an embodiment of the present disclosure the bacteriocin is at least one of pediocin PA-1 or bactofencin A.

In an aspect, the present disclosure relates to a kit comprising one or more bacteriocins or variants thereof and one or more applicators. In an embodiment of the present disclosure, the one or more bacteriocins or variants thereof are synthetic bacteriocins. In a further embodiment of the present disclosure, the synthetic bacteriocins or variants thereof are obtained by linear solid phase peptide synthesis. In a further embodiment of the present disclosure, the synthetic bacteriocins or variants thereof comprise a disulfide bond. In an embodiment of the present disclosure the bacteriocin is at least one of pediocin PA-1 or bactofencin A or variants thereof.

In an aspect, the present disclosure relates to a preservative comprising an effective amount of one or more bacteriocins in a physiological solution or in a food matrix, non-limiting examples of which include milk, yoghurt or cheese. In an embodiment of the present disclosure, the one or more bacteriocins are synthetic bacteriocins. In a further embodiment of the present disclosure, the synthetic bacteriocins are obtained by linear solid phase peptide synthesis. In a further embodiment of the present disclosure, the one or more synthetic bacteriocins comprise a disulfide bond. In a further embodiment of the present disclosure the one or more synthetic bacteriocins include at least one of pediocin PA-1 or bactofencin A.

In an aspect, the present disclosure relates to a preservative comprising an effective amount of one or more bacteriocins or variants thereof in a physiological solution or in a food matrix non-limiting examples of which include milk, yoghurt or cheese.. In an embodiment of the present disclosure, the one or more bacteriocins or variants thereof are synthetic bacteriocins or variants thereof. In a further embodiment of the present disclosure, the synthetic bacteriocins or variants thereof are obtained by linear solid phase peptide synthesis. In a further embodiment of the present disclosure, the one or more synthetic bacteriocins or variants thereof comprise a disulfide bond. In a further embodiment of the present disclosure the one or more synthetic bacteriocins include at least one of pediocin PA-1 or bactofencin A or variants thereof.

In an aspect, the present disclosure relates to a food packaging comprising an antimicrobial composition comprising an effective amount of one or more bacteriocins. In an embodiment of the present disclosure, the one or more bacteriocins are synthetic bacteriocins. In a further embodiment of the present disclosure, the synthetic bacteriocins are obtained by linear solid phase peptide synthesis. In a further embodiment of the present disclosure, the one or more synthetic bacteriocins comprise a disulfide bond. In a further embodiment of the present disclosure the one or more synthetic bacteriocins include at least one of pediocin PA-1 or bactofencin A. In yet a further embodiment of the present disclosure, the food packaging is a film, resin, liner, absorbent pad, plastic, shrink bag, shrink wrap, plastic wrap, Styrofoam™, carton, or cellulosic substrate.

In an aspect, the present disclosure relates to a food packaging comprising an antimicrobial composition comprising an effective amount of one or more bacteriocins or variants thereof. In an embodiment of the present disclosure, the one or more bacteriocins or variants thereof are synthetic bacteriocins or variants thereof. In a further embodiment of the present disclosure, the synthetic bacteriocins or variants thereof are obtained by linear solid phase peptide synthesis. In a further embodiment of the present disclosure, the one or more synthetic bacteriocins or variants thereof comprise a disulfide bond. In a further embodiment of the present disclosure the one or more synthetic bacteriocins include at least one of pediocin PA-1 or bactofencin A or variants thereof. In yet a further embodiment of the present disclosure, the food packaging is a film, resin, liner, absorbent pad, plastic, shrink bag, shrink wrap, plastic wrap, Styrofoam™, carton, or cellulosic substrate.

In an aspect, the present disclosure relates to a synthetic bacteriocin, non-limiting examples of which include bavaricin, bactofencin, helveticin, acidocin, lactocin, lactacin, lacticin, leucocin, lactococcin, pediocin, curvaticin, curvacin, mutacin, mesentericin, plantaricin, streptin or sakacin. In an embodiment of the present disclosure, the synthetic bacteriocin is obtained by linear solid phase peptide synthesis. In a further embodiment of the present disclosure, the synthetic bacteriocin comprises a disulfide bond.

In an aspect, the present disclosure relates to a synthetic bacteriocin or variants thereof, non-limiting examples of which include bavaricin, bactofencin, helveticin, acidocin, lactocin, lactacin, lacticin, nisin, leucocin, lactococcin, pediocin, curvaticin, curvacin, mutacin, mesentericin, plantaricin, streptin or sakacin or variants of any thereof. In an embodiment of the present disclosure, the synthetic bacteriocin or variants thereof is obtained by linear solid phase peptide synthesis. In a further embodiment of the present disclosure, the synthetic bacteriocin or variants thereof comprises a disulfide bond.

In an aspect, the present disclosure relates to synthetic gram-positive class II bacteriocins having a purity ranging from about 85% to about 99.9%. In an aspect, the present disclosure relates to synthetic gram-positive class IIa bacteriocins having a purity ranging from about 85% to about 99.9%. In an aspect, the present disclosure relates to synthetic gram-positive class IIb bacteriocins having a purity ranging from about 85% to about 99.9%. In an aspect, the present disclosure relates to synthetic gram-positive class IIc bacteriocins having a purity ranging from about 85% to about 99.9%. In an aspect, the present disclosure relates to synthetic gram-positive class IId bacteriocins having a purity ranging from about 85% to about 99.9%. In a further embodiment of the present disclosure, the synthetic gram-positive class II bacteriocins are prepared by solid phase peptide synthesis. In a further embodiment of the present disclosure, the synthetic gram-positive class IIa bacteriocins are prepared by linear solid phase peptide synthesis. In a further embodiment of the present disclosure, the synthetic gram-positive class IIb bacteriocins are prepared by linear solid phase peptide synthesis. In a further embodiment of the present disclosure, the synthetic gram-positive class IIc bacteriocins are prepared by linear solid phase peptide synthesis. In a further embodiment of the present disclosure, the synthetic gram-positive class IId bacteriocins are prepared by linear solid phase peptide synthesis. In a further embodiment of the present disclosure, the synthetic gram-positive class II bacteriocins comprise a disulfide bond. In a further embodiment of the present disclosure, the synthetic gram-positive class IIa bacteriocins comprise a disulfide bond. In a further embodiment of the present disclosure, the synthetic gram-positive class IIb bacteriocins comprise a disulfide bond. In a further embodiment of the present disclosure, the synthetic gram-positive class IIc bacteriocins comprise a disulfide bond. In a further embodiment of the present disclosure, the synthetic gram-positive class IId bacteriocins comprise a disulfide bond. In a further embodiment of the present disclosure, the disulfide bond is generated in situ.

In an aspect, the present disclosure relates to synthetic gram-positive class II bacteriocins or variants thereof having a purity ranging from about 85% to about 99.9%. In an aspect, the present disclosure relates to synthetic gram-positive class IIa bacteriocins or variants thereof having a purity ranging from about 85% to about 99.9%. In an aspect, the present disclosure relates to synthetic gram-positive class IIb bacteriocins or variants thereof having a purity ranging from about 85% to about 99.9%. In an aspect, the present disclosure relates to synthetic gram-positive class IIc bacteriocins or variants thereof having a purity ranging from about 85% to about 99.9%. In an aspect, the present disclosure relates to synthetic gram-positive class IId bacteriocins or variants thereof having a purity ranging from about 85% to about 99.9%. In a further embodiment of the present disclosure, the synthetic gram-positive class II bacteriocins or variants thereof are prepared by linear solid phase peptide synthesis. In a further embodiment of the present disclosure, the synthetic gram-positive class IIa bacteriocins or variants thereof are prepared by linear solid phase peptide synthesis. In a further embodiment of the present disclosure, the synthetic gram-positive class IIb bacteriocins or variants thereof are prepared by linear solid phase peptide synthesis. In a further embodiment of the present disclosure, the synthetic gram-positive class IIc bacteriocins or variants thereof are prepared by linear solid phase peptide synthesis. In a further embodiment of the present disclosure, the synthetic gram-positive class IId bacteriocins or variants thereof are prepared by linear solid phase peptide synthesis. In a further embodiment of the present disclosure, the synthetic gram-positive class II bacteriocins or variants thereof comprise a disulfide bond. In a further embodiment of the present disclosure, the synthetic gram-positive class IIa bacteriocins or variants thereof comprise a disulfide bond. In a further embodiment of the present disclosure, the synthetic gram-positive class IIb bacteriocins or variants thereof comprise a disulfide bond. In a further embodiment of the present disclosure, the synthetic gram-positive class IIc bacteriocins or variants thereof comprise a disulfide bond. In a further embodiment of the present disclosure, the synthetic gram-positive class IId bacteriocins or variants thereof comprise a disulfide bond. In a further embodiment of the present disclosure, the disulfide bond is generated in situ.

In an aspect, the present disclosure relates to the solid phase peptide synthesis of linear gram-positive class II bacteriocins. In an aspect, the present disclosure relates to the solid phase peptide synthesis of linear gram-positive class IIa bacteriocins. In an aspect, the present disclosure relates to the solid phase peptide synthesis of linear gram-positive class IIb bacteriocins. In an aspect, the present disclosure relates to the solid phase peptide synthesis of linear gram-positive class IIc bacteriocins. In an aspect, the present disclosure relates to the solid phase peptide synthesis of linear gram-positive class IId bacteriocins. In a further embodiment of the present disclosure, the linear gram-positive class II bacteriocins comprise a disulfide bond. In a further embodiment of the present disclosure, the linear gram-positive class IIa bacteriocins comprise a disulfide bond. In a further embodiment of the present disclosure, the linear gram-positive class IIb bacteriocins comprise a disulfide bond. In a further embodiment of the present disclosure, the linear gram-positive class IIc bacteriocins comprise a disulfide bond. In a further embodiment of the present disclosure, the linear gram-positive class IId bacteriocins comprise a disulfide bond. In a further embodiment of the present disclosure, the disulfide bond is generated in situ.

In an aspect, the present disclosure relates to the solid phase peptide synthesis of linear gram-positive class II bacteriocins or variants thereof. In an aspect, the present disclosure relates to the solid phase peptide synthesis of linear gram-positive class IIa bacteriocins or variants thereof. In an aspect, the present disclosure relates to the solid phase peptide synthesis of linear gram-positive class IIb bacteriocins or variants thereof. In an aspect, the present disclosure relates to the solid phase peptide synthesis of linear gram-positive class IIc bacteriocins or variants thereof. In an aspect, the present disclosure relates to the solid phase peptide synthesis of linear gram-positive class IId bacteriocins or variants thereof. In a further embodiment of the present disclosure, the linear gram-positive class II bacteriocins or variants thereof comprise a disulfide bond. In a further embodiment of the present disclosure, the linear gram-positive class IIa bacteriocins or variants thereof comprise a disulfide bond. In a further embodiment of the present disclosure, the linear gram-positive class IIb bacteriocins or variants thereof comprise a disulfide bond. In a further embodiment of the present disclosure, the linear gram-positive class IIc bacteriocins or variants thereof comprise a disulfide bond. In a further embodiment of the present disclosure, the linear gram-positive class IId bacteriocins or variants thereof comprise a disulfide bond. In a further embodiment of the present disclosure, the disulfide bond is generated in situ.

In an aspect, the present disclosure relates to the linear synthesis of gram-positive class IIa, IIb, IIc or IId bacteriocins. In an embodiment of the present disclosure, the synthesis comprises a solid phase synthesis of a linear peptide. In a further embodiment of the present disclosure, the synthesis comprises a solid phase synthesis of a linear peptide and chemical disulfide bond formation. In a further embodiment of the present disclosure, the synthesis comprises a solid phase synthesis of a linear peptide and in situ disulfide bond formation in a suitable matrix.

In an aspect, the present disclosure relates to the linear synthesis of gram-positive class IIa, IIb, IIc or IId bacteriocins or variants thereof. In an embodiment of the present disclosure, the synthesis comprises a solid phase synthesis of a linear peptide. In a further embodiment of the present disclosure, the synthesis comprises a solid phase synthesis of a linear peptide and chemical disulfide bond formation. In a further embodiment of the present disclosure, the synthesis comprises a solid phase synthesis of a linear peptide and in situ disulfide bond formation in a suitable matrix.

In an aspect, the present disclosure relates to in situ disulfide bond formation. In an embodiment of the present disclosure, disulfide bond formation comprises contacting a linear gram-positive class IIa bacteriocin comprising at least two sulfur containing amino acid residues with a bioassay medium or any other suitable medium. In an embodiment of the present disclosure, disulfide bond formation comprises contacting a linear gram-positive class IIb bacteriocin comprising at least two sulfur containing amino acid residues with a bioassay medium or any other suitable medium. In an embodiment of the present disclosure, disulfide bond formation comprises contacting a linear gram-positive class IIc bacteriocin comprising at least two sulfur containing amino acid residues with a bioassay medium or any other suitable medium. In an embodiment of the present disclosure, disulfide bond formation comprises contacting a linear gram-positive class IId bacteriocin comprising at least two sulfur containing amino acid residues with a bioassay medium or any other suitable medium.

In an aspect, the present disclosure relates to in situ disulfide bond formation. In an embodiment of the present disclosure, disulfide bond formation comprises contacting a linear gram-positive class IIa bacteriocin, or variants thereof, comprising at least two sulfur containing amino acid residues with a bioassay medium or any other suitable medium. In an embodiment of the present disclosure, disulfide bond formation comprises contacting a linear gram-positive class IIb bacteriocin, or variants thereof, comprising at least two sulfur containing amino acid residues with a bioassay medium or any other suitable medium. In an embodiment of the present disclosure, disulfide bond formation comprises contacting a linear gram-positive class IIc bacteriocin, or variants thereof, comprising at least two sulfur containing amino acid residues with a bioassay medium or any other suitable medium. In an embodiment of the present disclosure, disulfide bond formation comprises contacting a linear gram-positive class IId bacteriocin, or variants thereof, comprising at least two sulfur containing amino acid residues with a bioassay medium or any other suitable medium.

In an aspect, the present disclosure relates to a novel antimicrobial agents and uses thereof. In an embodiment, the present disclosure relates to the use of the novel antimicrobial agents for the prevention and/or treatment of bacterial related infections. In further embodiments of the present disclosure, the novel antimicrobial agents are used in applications related to food preservation and food safety. In further embodiments of the present disclosure, the novel antimicrobial agents comprise synthetic gram-positive class IIa, IIb, IIc or IId bacteriocins or variants thereof. In further embodiments of the present disclosure, the novel antimicrobial agents comprise synthetic gram-positive class IIa, IIb, IIc or IId bacteriocins, or variants thereof, comprising a disulfide bond. In a further embodiment of the present disclosure, the gram-positive class IIa, IIb, IIc or IId bacteriocins, or variants thereof, are obtained by linear solid phase peptide synthesis.

Also disclosed in the context of the present disclosure are embodiments 1 to 73. Embodiment 1 is a process for the linear synthesis of a bacteriocin or a variant thereof, the process comprising: stepwise addition of selected amino acids to a solid support; pseudoproline positioning and reopening; and cleavage of the bacteriocin or variant thereof from the solid support to provide a linear bacteriocin or variant thereof. Embodiment 2 is the process of embodiment 1, further comprising disulfide bond formation. Embodiment 2 is the process of embodiment 1 or 2, further comprising treating the linear bacteriocin with a mobile phase comprising an acid.

Embodiment 4 is a process for the linear synthesis of a gram-positive class II bacteriocin or a variant thereof, the process comprising: stepwise addition of selected amino acids to a solid support; pseudoproline positioning and reopening; cleavage of the gram-positive class II bacteriocin or the variant thereof from the solid support to provide a linear gram-positive class II bacteriocin or variant thereof; and disulfide bond formation. Embodiment 5 is the process of embodiment 4, further comprising treating the linear gram-positive class II bacteriocin or variant thereof with a mobile phase comprising an acid. Embodiment 6 is the process of any one of embodiments 2 to 5, wherein the disulfide bond formation comprises coupling a pair of thiol containing amino acid residues. Embodiment 7 is the process of embodiment 6, wherein the disulfide bond formation consists of chemically reacting the pair of thiol containing amino acid residues using an oxidant. Embodiment 8 is the process of embodiment 6, wherein the disulfide bond formation consists of in situ disulfide bond formation by contacting the linear bacteriocin with a suitable medium. Embodiment 9 is the process of embodiment 8, wherein the medium comprises at least one of a bioassay medium or a biological medium. Embodiment 10 is the process of any one of embodiments 6 to 9, wherein the thiol containing amino acids are at least one of cysteine, homocysteine or other amino acids bearing a thiol-containing side-chain. Embodiment 11 is the process of any one of embodiments 1 to 10, wherein the solid support comprises a ChemMatrix resin, a Wang resin, a polystyrene resin, a substituted polystyrene-based resin, a polyamide resin, a polyacrylate resin, a polyacrylamide resin and a polyethylene glycol-based resin. Embodiment 12 is the process of embodiment 11, wherein the solid support further comprises a resin linker. Embodiment 13 is the process of embodiment 12, wherein the resin linker is an HMPB linker, a Wang linker, a Rink amide linker, a PAL linker, a Ramage linker, a Sieber linker, a linker comprising an hydroxyl function or a trityl-based linker. Embodiment 14 is the process of any one of embodiments 3 to 5, wherein the acid comprises at least acetic acid. Embodiment 15 is the process of embodiment 4, wherein the gram-positive class II bacteriocin is a gram-positive class IIa bacteriocin or a variant thereof, a gram-positive class IIb bacteriocin or a variant thereof, a gram-positive class IIc bacteriocin or a variant thereof or a gram-positive class IId bacteriocin or a variant thereof. Embodiment 16 is the process of embodiment 15, wherein the gram-positive class IIa bacteriocin or variant thereof is a pediocin-like bacteriocin or variant thereof. Embodiment 17 is the process of embodiment 15, wherein the gram-positive class IId bacteriocin or variant thereof is a bactofencin-like bacteriocin or variant thereof. Embodiment 18 is the process of any one of embodiments 4 to 17, wherein the variant has at least 80% sequence identity with an unmodified or native reference sequence. Embodiment 19 is the process of any one of embodiments 4 to 18, wherein the variant has at least 85% sequence identity with an unmodified or native reference sequence. Embodiment 20 is the process of any one of embodiments 4 to 19, wherein the variant has at least 90% sequence identity with an unmodified or native reference sequence. Embodiment 21 is the process of any one of embodiments 4 to 20, wherein the variant has at least 95% sequence identity with an unmodified or native reference sequence. Embodiment 22 is the process of any one of embodiments 4 to 21, wherein the variant has at least 99% sequence identity with an unmodified or native reference sequence. Embodiment 23 is the process of any one of embodiments 4 to 17, wherein the variant has at least one of an amino acid substitution, modification, addition or deletion relative to an unmodified or native reference sequence. Embodiment 24 is the process of embodiment 23, comprising at least two amino acid substitutions. Embodiment 25 is the process of embodiment 23, comprising at least three amino acid substitutions. Embodiment 26 is the process of embodiment 23, comprising at least four amino acid substitutions. Embodiment 27 is the process of any one of embodiments 23 to 26, wherein the amino acid substitution comprises substituting at least one of arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine and tryptophan for any one of arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine and tryptophan. Embodiment 28 is the process of embodiment 27, wherein the amino acid substitution comprises substituting at least one of arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine and tryptophan for alanine, leucine, phenylalanine, tryptophan, or serine. Embodiment 29 is the process of embodiment 28, wherein the amino acid substitution comprises substituting at least one of lysine, arginine, histidine, cysteine, valine, tyrosine, asparagine, glycine, methionine, proline, threonine, tryptophan and cysteine for alanine. Embodiment 30 is the process of embodiment 29, wherein the amino acid substitution comprises substituting methionine for leucine. Embodiment 31 is the process of embodiment 29, wherein the amino acid substitution comprises substituting tyrosine for phenylalanine, serine or tryptophan.

Embodiment 32 is a process for the linear synthesis of a gram-positive class II bacteriocin or a variant thereof, the process comprising: stepwise addition of selected amino acids to a solid support; pseudoproline positioning and reopening; cleavage of the gram-positive class II bacteriocin or the variant thereof from the solid support to provide a linear gram-positive class II bacteriocin or variant thereof; and in situ disulfide bond formation. Embodiment 33 is the process of embodiment 32, further comprising treating the linear gram-positive class II bacteriocin or variant thereof with a mobile phase comprising an acid. Embodiment 34 is the process of embodiment 32 or 33, wherein the in situ disulfide bond formation comprises coupling a pair of thiol containing amino acid residues. Embodiment 35 is the process of any one of embodiments 32 to 35, wherein the in situ disulfide bond formation comprises contacting the linear bacteriocin with a suitable medium. Embodiment 36 is the process of embodiment 35, wherein the medium comprises at least one of a bioassay medium or a biological medium. Embodiment 37 is the process of any one of embodiments 34 to 36, wherein the thiol containing amino acids are at least one of cysteine, homocysteine or other amino acids bearing a thiol-containing side-chain. Embodiment 38 is the process of any one of embodiments 32 to 37, wherein the solid support comprises a ChemMatrix resin, a Wang resin, a polystyrene resin, a substituted polystyrene-based resin, a polyamide resin, a polyacrylate resin, a polyacrylamide resin and a polyethylene glycol-based resin. Embodiment 39 is the process of embodiment 38, wherein the solid support further comprises a resin linker. Embodiment 40 is the process of embodiment 39, wherein the resin linker is an HMPB linker, a Wang linker, a Rink amide linker, a PAL linker, a Ramage linker, a Sieber linker, a linker comprising an hydroxyl function or a trityl-based linker. Embodiment 41 is the process of any one of embodiments 33 to 40, wherein the acid comprises at least acetic acid. Embodiment 42 is the process of any one of embodiments 32 to 41, wherein the gram-positive class II bacteriocin is a gram-positive class IIa bacteriocin or a variant thereof, a gram-positive class IIb bacteriocin or a variant thereof, a gram-positive class IIc bacteriocin or a variant thereof or a gram-positive class IId bacteriocin or a variant thereof. Embodiment 43 is the process of embodiment 42, wherein the gram-positive class IIa bacteriocin or variant thereof is a pediocin-like bacteriocin or variant thereof. Embodiment 44 is the process of embodiment 42, wherein the gram-positive class IId bacteriocin or variant thereof is a bactofencin-like bacteriocin or variant thereof. Embodiment 45 is the process of any one of embodiments 32 to 44, wherein the variant has at least 80% sequence identity with an unmodified or native reference sequence. Embodiment 46 is the process of any one of embodiments 32 to 45, wherein the variant has at least 85% sequence identity with an unmodified or native reference sequence. Embodiment 47 is the process of any one of embodiments 32 to 46, wherein the variant has at least 90% sequence identity with an unmodified or native reference sequence. Embodiment 48 is the process of any one of embodiments 32 to 47, wherein the variant has at least 95% sequence identity with an unmodified or native reference sequence. Embodiment 49 is the process of any one of embodiments 32 to 48, wherein the variant has at least 99% sequence identity with an unmodified or native reference sequence. Embodiment 50 is the process of any one of embodiments 32 to 44, wherein the variant has at least one of an amino acid substitution, modification, addition or deletion relative to an unmodified or native reference sequence. Embodiment 51 is the process of embodiment 50, comprising at least two amino acid substitutions. Embodiment 52 is the process of embodiment 50, comprising at least three amino acid substitutions. Embodiment 53 is the process of embodiment 50, comprising at least four amino acid substitutions. Embodiment 54 is the process of any one of embodiments 50 to 53, wherein the amino acid substitution comprises substituting at least one of arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine and tryptophan for any one of arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine and tryptophan. Embodiment 55 is the process of embodiment 54, wherein the amino acid substitution comprises substituting at least one of arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine and tryptophan for alanine, leucine, phenylalanine, tryptophan, or serine. Embodiment 56 is the process of embodiment 55, wherein the amino acid substitution comprises substituting at least one of lysine, arginine, histidine, cysteine, valine, tyrosine, asparagine, glycine, methionine, proline, threonine, tryptophan and cysteine for alanine. Embodiment 57 is the process of embodiment 55, wherein the amino acid substitution comprises substituting methionine for leucine. Embodiment 58 is the process of embodiment 55, wherein the amino acid substitution comprises substituting tyrosine for phenylalanine, serine or tryptophan.

Embodiment 59 is a composition comprising a bacteriocin as obtained by the process of any one of embodiments 1 to 58 and a pharmacologically acceptable carrier. Embodiment 60 is a method for the prevention and/or treatment of bacterial related infections comprising administering to a subject in need thereof an effective amount of a bacteriocin or a variant thereof as obtained by the process of any one of embodiments 1 to 58. Embodiment 61 is a method of preserving a food item comprising applying an effective amount of a bacteriocin or a variant thereof as obtained by the process of any one of embodiments 1 to 58 to the food item. Embodiment 62 is the method of embodiment 61, wherein the bacteriocin is applied to the surface or within the food item. Embodiment 63 is a bactericidal food preservation composition adapted for coating a food product for preservation thereof, the composition comprising a bacteriocin or a variant thereof as obtained by the process of any one of embodiments 1 to 58 in an amount effective to kill a pathogenic agent upon contact. Embodiment 64 is an item comprising a surface, wherein the surface comprises an antimicrobial effective amount of a bacteriocin as obtained by the process of any one of embodiments 1 to 58. Embodiment 65 is the item of embodiment 64, wherein the item is selected from the group consisting of a medical device, medical instrument and medical implement. Embodiment 66 is a kit comprising one or more bacteriocins or variants thereof as obtained by the process of any one of claims 1 to 58 and one or more applicators. Embodiment 67 is a preservative comprising an effective amount of one or more bacteriocins or variants thereof as obtained by the process of any one of embodiments 1 to 58 in a physiological solution. Embodiment 68 is a food packaging comprising an antimicrobial composition comprising an effective amount of one or more bacteriocins or variants thereof as obtained by the process of any one of embodiments 1 to 58. Embodiment 69 is the food packaging of embodiment 68, wherein the food packaging is a film, resin, liner, absorbent pad, plastic, shrink bag, shrink wrap, plastic wrap, Styrofoam™, carton, or cellulosic substrate. Embodiment 70 is the process of any one of embodiments 1 to 58, wherein the bacteriocin or variant thereof is at least one of bavaricin, helveticin, acidocin, lactocin, lactacin, lacticin, leucocin, lactococcin, pediocin, curvaticin, curvacin, mutacin, mesentericin, plantaricin, streptin, sakacin or variants thereof. Embodiment 71 is a synthetic gram-positive class II bacteriocin or a variant thereof, wherein the synthetic gram-positive class II bacteriocin is obtained by linear solid phase peptide synthesis. Embodiment 72 is the synthetic gram-positive class II bacteriocin of embodiment 71, wherein the synthetic gram-positive class II bacteriocin is pediocin PA-1 or variants thereof. Embodiment 73 is the synthetic gram-positive class II bacteriocin of embodiment 71, wherein the synthetic gram-positive class II bacteriocin is bactofencin A or variants thereof.

Without wishing to be bound by theory, it is believed that the in situ disulfide bond formation occurs in the linear bacteriocin(s) of the present invention due to the presence of disulfide bond containing proteins present in the food items (e.g., food items such as pork, salmon, ham, milk, cream, sausages, mushrooms, milk, yogurt, cheese, etc.). In particular, a food item includes a food matrix. A food matrix includes proteins having disulfide bonds. The disulfide bonds in the proteins in the food matrix allow for thiol containing amino acids in the linear bacteriocin(s) of the present invention to form a disulfide bond. This occurs by a bimolecular thio-disulfide exchange mechanism (S_{N}2 reaction mechanism) between free thiol groups contained in the linear bacteriocin and the disulfide containing proteins from the food matrix.

The foregoing and other advantages and features of the present disclosure will become more apparent upon reading of the following non-restrictive description of illustrative embodiments thereof, given by way of example only with reference to the accompanying drawings/figures.

### BRIEF DESCRIPTION OF THE DRAWINGS/FIGURES

In the appended drawings/figures:

FIG. 1 illustrates the HPLC profile of linear pediocin PA-1 1 as obtained following removal from the solid support in accordance with an embodiment of the present disclosure.

FIGS. 2A-C illustrates the HPLC and ESI-MS profiles of: FIG. 2A) crude oxidized pediocin PA-1 2a, 2b and 2c; FIG. 2B) purified pediocin PA-1 3c; and FIG. 2C) the MALDI-TOF MS spectrum of synthetic pediocin PA-1 3c in accordance with an embodiment of the present disclosure.

FIGS. 3A-F illustrate a soft agar TSBY diffusion growth inhibition assay for native pediocin PA-1 and synthetic variant peptides 2c, 3a, 3b, 3c, 4, 6 against L. monocytogenes LSD530. FIGS. 3A-E: 80 µL of supernatant from pediocin PA-1 producing P. acidilacti UL5 (24 mm) and 80 µL of a 1 mg/mL solution of: FIG. 3A) 1 (32 mm); FIG. 3B) 2c (14 mm); 3c (31 mm) and 6 (n/a); C) 3a (24 mm) and 3b (27 mm); FIG. 3D) 4 (31 mm); and FIG. 3E) 5 (34 mm). FIG. 3F illustrates a soft agar MRS diffusion growth inhibition assay for 80 µL of supernatant from Nisin producing L. lactis ATCC 11454 (18 mm) and 80 µL of synthetic peptides 2c, 3a, 3b, 3c, 4, 6 against P. acidilacti UL5.

FIG. 4 illustrates the antimicrobial activity of different concentrations of the linear pediocin PA-1 analog 4 in skim milk (10 mL) against L. monocytogenes ATCC19111 at 30° C. The average bacterial growth (CFU/mL) for 12 h is shown.

FIGS. 5A-B illustrates circular dichroism spectra obtained for purified pediocin PA-1 3c, linear pediocin PA-1 1 and analogs 2c and 6: FIG. 5A) 3c in aqueous TFE solutions (0, 25, 50, 75 or 90% TFE in H₂O) and FIG. 5B) 1, 2c, 3c, and 6 in DMPC (left) and DMPG (right) phospholipid vesicles (lipid/peptide ratio of 10:1).

FIGS. 6A-C illustrates the lowest relative energy structure (α-helix red, β-sheet yellow, loops green and disulfide bridge orange) for synthetic pediocin PA-1 analog 5 as determined by ¹H NMR spectroscopy (50/50 H₂O/TFE-d2 at 313°K) (FIG. 6A and FIG. 6B); the lowest energy structures aligned with helix (T23 to T35) (FIG. 6C).

FIGS. 7A-C illustrates the HPLC and ESI-MS profiles of: FIG. 7A) linear sequence bactofencin A and synthetic variant peptide 2 (Table 6); FIG. 7B) soft agar TSBY diffusion growth inhibition assay for linear sequence bactofencin A and synthetic variant peptides 2, 3 and 4 against S. aureus ATCC 6538, and linear sequence bactofencin A and synthetic variant peptide 2 against L. monocytogenes ATCC 19111; FIG. 7C) activity profiles at various concentrations for linear sequence bactofencin A (left) and synthetic variant peptide 2 (right) against L. monocytogenes. ATCC 19111.

FIG. 8 illustrates a portion of the phylogenetic tree demonstrating that Clostridium botulinum, like Listeria monocytogenes, possesses the PTS mannose transporter subunit IID recognized by Pediocin PA-1 M31L (5).

FIG. 9 illustrates the characteristic black halo of Listeria monocytogenes on PALCAM agar.

FIG. 10, and with reference to Table 10, illustrates the enumeration of Listeria monocytogenes on diced pork tenderloin over 7 days at 4° C. Batch 1: negative control, batch 2: positive control, batch 3: 150 ng/g of Pediocin PA-1 M31L (5) treatment **** = p-value < 0.0001.

FIG. 11, and with reference to Table 11, illustrates the enumeration of Listeria monocytogenes on diced fresh salmon filet over 7 days at 4° C. Batch 1: negative control, batch 2: positive control, batch 3: 150 ng/g of Pediocin PA-1 M31L (5) treatment **** = p-value < 0.0001. * = p-value < 0.05.

FIG. 12, and with reference to Table 12, illustrates the enumeration of Listeria monocytogenes on cold-smoked salmon over 21 days at 4° C., vacuumed-packed. Batch 1: negative control, batch 2: positive control, batch 3: 150 ng/g of Pediocin PA-1 M31L (5) treatment and batch 4: 50 ng/g of Pediocin PA-1 M31L (5) treatment. **** = p-value < 0.0001.

FIG. 13, and with reference to Table 13, illustrates the enumeration of Listeria monocytogenes on sliced ham over 10 days of incubation at 4° C. **** means a p value < 0.0001.

FIG. 14, and with reference to Table 14, illustrates the enumeration of Listeria monocytogenes (cfu/ml, bottom portion; and Log, top portion) in raw milk for each day of analysis.

FIG. 15, and with reference to Table 15, illustrates the enumeration of Listeria monocytogenes (cfu/ml, bottom portion; and Log, top portion) in raw cream for each day of analysis.

FIG. 16, and with reference to Table 16, illustrates the enumeration of Listeria monocytogenes (cfu/g, bottom portion; and Log, top portion) of sausages for each experimental group at each analysis day. Statistical values ∗∗∗∗ show that using pediocin PA-1 M31L (5) at 150 ppb significantly reduces the amount of Listeria monocytogenes compared to the positive control with a p value < 0.0001.

FIG. 17, and with reference to Tables 17 and 18, illustrates the enumeration of Listeria monocytogenes (cfu/g, bottom portion; and Log, top portion) for each sampling day on sliced mushrooms (n=3).

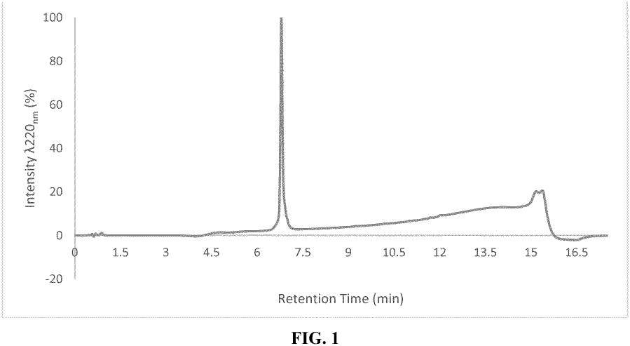

### DETAILED DESCRIPTION

### Glossary

In order to provide a clear and consistent understanding of the terms used in the present disclosure, a number of definitions are provided below. Moreover, unless defined otherwise, all technical and scientific terms as used herein have the same meaning as commonly understood by one of ordinary skill in the art to which this disclosure pertains.

The word “a” or “an” when used in conjunction with the term “comprising” in the claims and/or the disclosure may mean “one”, but it is also consistent with the meaning of “one or more”, “at least one”, and “one or more than one” unless the content clearly dictates otherwise. Similarly, the word “another” may mean at least a second or more unless the content clearly dictates otherwise.

As used in this disclosure and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or openended and do not exclude additional, unrecited elements or process steps.

As used in this disclosure and claim(s), the word “consisting” and its derivatives, are intended to be close ended terms that specify the presence of stated features, elements, components, groups, integers, and/or steps, and also exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of these features, elements, components, groups, integers, and/or steps.

The terms “about”, “substantially” and “approximately” as used herein mean a reasonable amount of deviation of the modified term such that the end result is not significantly changed. These terms of degree should be construed as including a deviation of at least ±1% of the modified term if this deviation would not negate the meaning of the word it modifies.

The term “suitable” as used herein means that the selection of the particular compound (e.g. amino acid) and/or reagent (e.g. coupling reagent) and/or conditions would depend on the specific manipulation to be performed, but the selection would be well within the skill of a person trained in the art. All process/method steps described herein are to be conducted under conditions sufficient to provide the product (e.g. peptide) shown. A person skilled in the art would understand that all process/method conditions, including, for example, process/method solvent, process/method time, process/method temperature, process/method pressure, reagent/ingredient ratio and whether or not the process/method should be performed under an anhydrous or inert atmosphere, can be varied to optimize the yield of the desired product and it is within their skill to do so.

The expression “proceed to a sufficient extent” as used herein with reference to the process/method steps disclosed herein means that the process/method steps proceed to an extent that conversion of the starting material or substrate to product is maximized. Conversion may be maximized when greater than about 5, 10, 15, 20, 25, 30, 35, 40, 45, 50, 55, 60, 65, 70, 75, 80, 85, 90, 95 or 99% of the starting material or substrate is converted to product.

The term “analogue” as used herein with reference to the antibacterial peptides refers to a peptide in which one or more individual atoms or functional groups or amino acid residues have been replaced, either with a different atom or a different functional group or a different amino acid residue.

The term “chemical modification” refers to a change in the naturally-occurring chemical structure of one or more amino acids of a polypeptide. Such modifications can be made to a side chain or a terminus, e.g., changing the amino-terminus or carboxyl terminus. In some embodiments, the modifications are useful for creating chemical groups that can conveniently be used to link the polypeptides to other materials.

The term “amino acid” as used herein refers to an organic acid containing both a basic amino group and an acidic carboxyl group. Therefore, the molecule is amphoteric and exists in aqueous solution as dipole ions. In an embodiment of the present disclosure, the amino acids are the L-amino acids. They include but are not limited to the 25 amino acids that have been established as protein constituents. They must contain at least one carboxyl group and one primary or secondary amino group on the amino acid molecule. They include such proteinogenic amino acids as alanine, valine, leucine, isoleucine, norleucine, proline, hydroxyproline, phenylalanine, tryptophan, methionine, glycine, serine, threonine, cysteine, tyrosine, asparagine, glutamine, aspartic acid, glutamic acid, lysine, hydroxylysine, ornithine, arginine, histidine, penicillamine and the like. In an embodiment of the present disclosure, the amino acids are the D-amino acids. In an embodiment of the present disclosure, the amino acids are a mixture of the L- and the D-amino acids.

The term “resin linker” as used herein refers to a molecule attached to the solid support for connecting the peptide chain to the solid support. Linker molecules are generally designed such that eventual cleavage provides either a free acid or amide at the C-terminus. Linkers are generally not resin-specific. The first amino acid of the peptide sequence may be attached to the linker after the linker is attached to the solid support or attached to the solid support using a linker that includes the first amino acid of the peptide sequence.

Conservative changes can generally be made to an amino acid sequence without altering activity. These changes are termed “conservative substitutions”; that is, an amino acid belonging to a grouping of amino acids having a particular size or characteristic can be substituted for another amino acid. Substitutes for an amino acid sequence can be selected from other members of the class to which the amino acid belongs. For example, the nonpolar (hydrophobic) amino acids include alanine, leucine, isoleucine, valine, proline, phenylalanine, tryptophan, methionine, and tyrosine. The polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Such substitutions are not expected to substantially affect apparent molecular weight as determined by polyacrylamide gel electrophoresis or isoelectric point. Conservative substitutions also include substituting optical isomers of the sequences for other optical isomers, specifically d amino acids for l amino acids for one or more residues of a sequence. Moreover, all of the amino acids in a sequence can undergo a d to l isomer substitution. Exemplary conservative substitutions include, but are not limited to, Lys for Arg and vice versa to maintain a positive charge; Glu for Asp and vice versa to maintain a negative charge; Ser for Thr so that a free —OH is maintained; and Gln for Asn to maintain a free —NH₂. Yet another type of conservative substitution constitutes the case where amino acids with desired chemical reactivities are introduced to impart reactive sites for chemical conjugation reactions, if the need for chemical derivatization arises. Such amino acids include but are not limited to Cys (to insert a sulfhydryl group), Lys (to insert a primary amine), Asp and Glu (to insert a carboxylic acid group). Moreover, substitutions, deletions and insertions of the polypeptide sequences can in some cases be made without a loss of function of the polypeptide. Substitutions can include, e.g., 1, 2, 3, 4, 5, 6, 7, 8, 9, 10, 15 or 20 residues (including any number of substitutions between those listed). A variant of a particular synthetic bacteriocin may exhibit a total number of up to 20 (e.g., up to 1, 2, 3, 4, 5, 6, 7, 8, 9, 10, 15 or 20, including any number in between those listed) changes (e.g., substitutions, deletions, N-terminal and/or C-terminal modifications) in the in the amino acid sequence. In particular embodiments, the variants exhibit about 80%, about 85%, about 90%, about 95%, about 97%, about 98%, about 99% functional equivalence to an unmodified or native reference sequence. The amino acid residues described herein employ either the single letter amino acid designator or the three-letter abbreviation in keeping with the standard polypeptide nomenclature. All amino acid residue sequences are represented herein by formulae with left and right orientation in the conventional direction of amino-terminus to carboxy-terminus.

The term “sequence identity” is used with regard to polypeptide sequence comparisons. This expression in particular refers to a percentage of sequence identity, for example at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% to the respective reference polypeptide. Particularly, the polypeptide in question and the reference polypeptide exhibit the indicated sequence identity over a continuous stretch of 10, 20, 30, 40, 45, 50, 60, 70, 80, 90, 100 or more amino acids or over the entire length of the reference polypeptide.

In an aspect, the present disclosure relates to the linear synthesis of gram-positive class IIa, IIb, IIc or IId bacteriocins or variants thereof. In an embodiment of the present disclosure, the synthesis comprises a linear solid phase synthesis of gram-positive class IIa, IIb, IIc or IId bacteriocins or variants thereof. In a further aspect, the present disclosure relates to synthetic gram-positive class IIa, IIb, IIc or IId bacteriocins or variants thereof. In yet a further aspect, the present disclosure relates to synthetic gram-positive class IIa, IIb, IIc or IId bacteriocins, or variants thereof, comprising a disulfide bond.

In certain embodiments, the synthetic gram-positive class IIa, IIb, IIc or IId bacteriocin is, is at least, or is at most 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 amino acids in length (or any range derivable therein).

In an aspect, the present disclosure relates to a process for the linear synthesis of gram-positive class IIa bacteriocins and compositions and uses thereof. In an embodiment, the present disclosure relates to the linear synthesis of pediocin PA-1 and compositions and uses thereof. In a further embodiment of the present disclosure, the synthesis of gram-positive class IIa bacteriocins comprises the use of linear solid phase peptide synthesis. In yet a further embodiment of the present disclosure, the process for the synthesis of pediocin PA-1 comprises the use of linear solid phase peptide synthesis. In yet a further embodiment of the present disclosure, the process for the synthesis of bactofencin A comprises the use of linear solid phase peptide synthesis. In yet a further embodiment of the present disclosure, the various peptides and analogues thereof, were prepared by linear solid phase peptide synthesis using the Fmoc/t-Bu strategy on a HMPB-ChemMatrix® solid support.

The linear synthesis of bacteriocins obviates the need for disulfide formation prior to use. Indeed, large amounts of linear bacteriocins could be produced in high yields at least in view of the absence of a synthetic oxidation step (disulfide bond formation) and an associated purification step. Moreover, the linear synthesis allows for the ready substitution of any amino acid and thus the synthesis of a great many variants of a given bacteriocin.

In an aspect, the present disclosure relates to the linear solid phase peptide synthesis of pediocin PA-1 and variants thereof. It is to be understood that all process/method steps described herein are to be conducted under conditions sufficient to provide the desired end product (e.g. a gram-positive class IIa bacteriocin). A person skilled in the art would understand that all processing conditions, including, for example, processing time, processing temperature, and whether or not the process should be performed under an anhydrous or inert atmosphere, can be varied to optimize the yield of the desired product and it is within their skill to do so.

It will be apparent to one skilled in the art, that in the course of peptide synthesis, it may be necessary to protect certain side chains of the amino acids to prevent unwanted side reactions. For example, it may be necessary to protect the hydroxyl group on the side chain of tyrosine, serine, or threonine in order to prevent these groups from interfering with the desired reactions. This is a common problem in peptide synthesis and many procedures are available for protecting the functional groups on the side chains of the amino acids. Such procedures for protecting various functional groups are known to one skilled in the art and are described in the treatise entitled “PEPTIDES: CHEMISTRY AND BIOLOGY”, Norbert Sewald and Hans-Dieter Jakubke, 2^{nd} Edition, Wiley-VCH Verlag GmbH & Co., Weinheim, 2009, and the reference book Protective Groups in Organic Synthesis, 3^{rd} Edition, by T.W. Green and P.G.M. Wuts, John Wiley and Sons, New York, 2002, the contents of both being incorporated herein by reference.

In accordance with an embodiment of the present disclosure, and with reference to Scheme 1, there is shown a linear solid phase peptide synthesis of pediocin PA-1. The HMPB-ChemMatrix® solid support was selected to perform the synthesis in view of its higher performance with larger peptides and/or its ability to form aggregation-disrupting interactions with the growing peptide chains. An initial attempt at preparing the linear precursor 1 by stepwise amino acid additions yielded a complex mixture of short peptides while the desired peptide product could not be observed. In order to identify the problematic amino acid couplings, a series of C-terminal ladder sequences starting from Gly40 and working upstream (GNHKC, QGNHKC, etc.) were prepared in parallel and analyzed by HPLC-MS after their cleavage from the resin. The results showed that the coupling of Fmoc-Ala-OH on Gly29 was ineffective. As reported in a previous study on pediocin analogs, pseudoprolines were incorporated to address these problematic couplings by coupling Fmoc-Val-Thr(Ψ^{Me,Me}pro)-OH on residue Cys9, and Fmoc-Ala-Thr(Ψ^{Me,Me}pro)-OH on residues Thr23 and Gly36. [21]. The combined use of the HMPB linker and the ChemMatrix solid support (resin), as well as the positioning of the aforementioned pseudoprolines, provides for the preparation and isolation of linear pediocin PA-1 1 following side-chain deprotection and cleavage from the resin. In an embodiment of the present disclosure, peptide cleavage from the resin is achieved by exposing the resin-bound peptides to a TFA cocktail over a period of time sufficient to yield the crude peptide. In yet a further embodiment of the present disclosure, the resin-bound peptide is contacted with the TFA cocktail over a period ranging from 1 to 5 hours. In yet a further embodiment of the present disclosure, the resin-bound peptide is contacted with the TFA cocktail over a period of 3 hours. In yet a further embodiment of the present disclosure, the peptide is contacted with a second TFA cocktail over a period ranging from 1-3 hours. The crude linear pediocin PA-1 1 was isolated in >90% crude purity. In an embodiment of the present disclosure, the TFA cocktail comprises TFA/TIPS/H₂O (95:2.5:2.5).

In addition to the desired peptide product, HPLC-MS analysis of the crude peptide product illustrated that side-chain alkylated peptides represent a significant source of impurity. It was fortuitously discovered that an initial treatment of the resin-bound peptide with the TFA cocktail, followed by precipitating the released crude peptide in diethyl ether and a further treatment with TFA cocktail substantially avoided the formation of these unwanted alkylated adducts. This observation was corroborated by further HPLC analysis showing only a single signal that was attributed to the desired linear pediocin PA-1 1 (55-70% overall yield).

In a particular embodiment of the present disclosure, the synthetic linear gram-positive class II bacteriocins have a purity ranging from about 85% to about 99.9%. In further embodiments of the present disclosure, the synthetic linear gram-positive class II bacteriocins have a purity of at least 85%, 86%, 87%, 88%, 89%, 90%, 91%, 92%, 93%, 94%, 95%, 96%, 97%, 98% or 99% or any range derivable therein. In a particular embodiment of the present disclosure, the synthetic linear gram-positive class IIa bacteriocins have a purity ranging from about 85% to about 99.9%. In further embodiments of the present disclosure, the synthetic linear gram-positive class IIa bacteriocins have a purity of at least 85%, 86%, 87%, 88%, 89%, 90%, 91%, 92%, 93%, 94%, 95%, 96%, 97%, 98% or 99% or any range derivable therein. In a particular embodiment of the present disclosure, the synthetic linear gram-positive class IIb bacteriocins have a purity ranging from about 85% to about 99.9%. In further embodiments of the present disclosure, the synthetic linear gram-positive class IIb bacteriocins have a purity of at least 85%, 86%, 87%, 88%, 89%, 90%, 91%, 92%, 93%, 94%, 95%, 96%, 97%, 98% or 99% or any range derivable therein. In further embodiments of the present disclosure, the synthetic linear gram-positive class IIc bacteriocins have a purity of at least 85%, 86%, 87%, 88%, 89%, 90%, 91%, 92%, 93%, 94%, 95%, 96%, 97%, 98% or 99% or any range derivable therein. In further embodiments of the present disclosure, the synthetic linear gram-positive class IId bacteriocins have a purity of at least 85%, 86%, 87%, 88%, 89%, 90%, 91%, 92%, 93%, 94%, 95%, 96%, 97%, 98% or 99% or any range derivable therein.

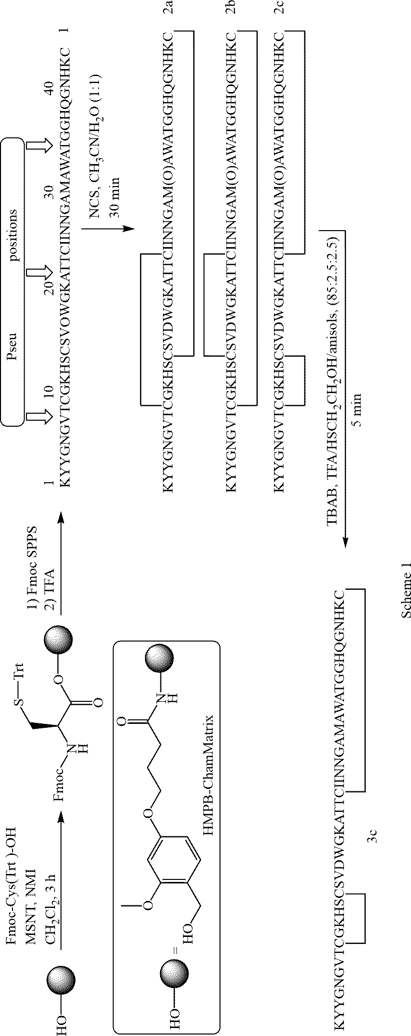

Formation of selective disulfide linkages under mild oxidative conditions generated PA-1 3. Disulfide bond formation however proved to be a challenge in view of the Met31 being very sensitive to oxidation. It was observed that PA-1 2, comprising disulfide linkages between Cys9-Cys24 and Cys14-Cys44 (2a), between Cys9-Cys44 and Cys14-Cys24 (2b), and between Cys9-Cys14 and Cys24-Cys24 (2c), showed no activity. This lack of activity is attributed to the presence of the oxidized Met31 residue. To address the problem associated with the concomitant oxidation of the Met31 residue, it was fortuitously discovered the desired PA-1 3 could be obtained by simultaneous disulfide bond formation and Met31 oxidation using N-chlorosuccinimide (NCS), followed by selective reduction of the Met31 residue using TBAB/TFA/HSCH₂CH₂OH/Anisole.

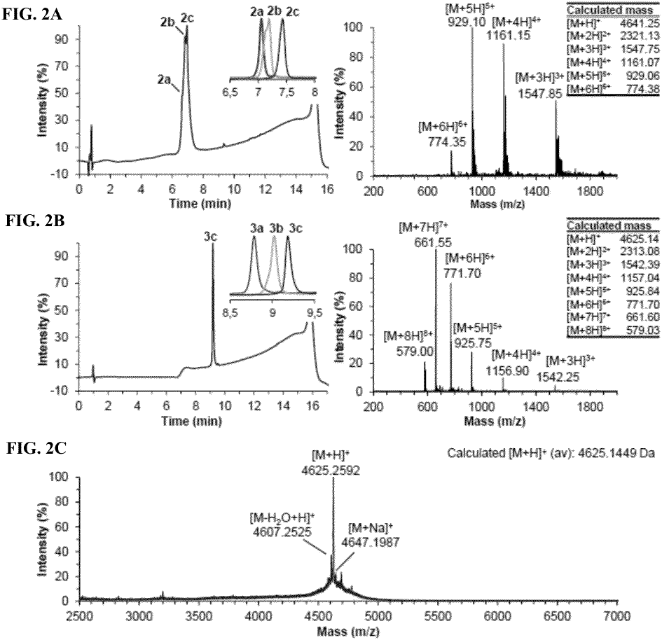

Initial attempts at the formation of PA-1 2 from linear precursor PA-1 1 using NCS, resulted in a great number of side products as observed by HPLC. Further analysis of the associated MS and MS/MS spectra revealed that most of the side products comprise peptides having chlorinated aromatic side chains. The presence of such peptide side-products was attributed to the presence of residual TFA counter ions in the purified linear precursor PA-1 1. Such TFA counter ions have been previously reported as catalysts for the chlorination of aromatic residues using NCS. Further purification of the linear precursor PA-1 1 by HPLC, using acetic acid in the mobile phase, resulted in the substantially complete removal of any TFA counter ions from the linear precursor PA-1 1. Indeed, when PA-1 1 was subjected to simultaneous disulfide bond formation and Met31 oxidation using N-chlorosuccinimide (NCS), no chlorinated adducts could be observed. PA-1 2 was obtained as a mixture of 2a (7.5%), 2b (21.5%) and 2c (71.0%) (as determined by HPLC) (FIG. 2A). Subsequent purification by HPLC and MS/MS analysis of the three isolated peptides, provided for the identification of the third peak as that of the oxidized native pediocin PA-1 2c.[23] Reduction of the oxidized Met31 residue at room temperature using tetrabutylammonium bromide (TBAB) in TFA, in the presence of mercaptoethanol (HSCH₂CH₂OH) and anisole, yielded PA-1 3c. The reduction of the oxidized Met31 residue could be achieved without affecting any of the previously established disulfide bonds. Crude pediocin PA-1 3c was subsequently purified by precipitation in cold diethyl ether.

The presence of synthetic pediocin PA-1 3c was confirmed by HPLC-MS analysis (FIG. 2B) and MALDI-TOF MS (FIG. 2C). MALDI-TOF MS analysis corroborated the presence of synthetic pediocin PA-1 3c as per the observation of a molecular ion at 4625.2592 Da (calculated [M+H]+ for C₁₉₆H₂₉₄N₆₁O₆₀S₅: 4625.1449 Da). The amino acid sequence of synthetic pediocin PA-1 3c was subsequently validated by MS/MS following disulfide bond reduction, cysteine S-alkylation using iodoacetamide and trypsin digestion. A similar synthetic process was applied for the synthesis of pediocin PA-1 3a and pediocin PA-1 3b from purified PA-1 2a and PA-1 2b respectively. Table 3 illustrates the peptides synthesized.

TABLE 3

Peptides synthesized
ID Sequence

| | |
|---|---|
| 1 | KYYGNGVTCGKHSCSVDWGKATTCIINNGAMAWATGGHQGNHKC |
| 2c | KYYGNGVTCGKHSCSVDWGKATTCIINNGAM(O)AWATGGHQGNHKC |
| 3a | KYYGNGVTCGKHSCSVDWGKATTCIINNGAMAWATGGHQGNHKC |
| 3b | KYYGNGVTCGKHSCSVDWGKATTCIINNGAMAWATGGHQGNHKC |
| 3c | KYYGNGVTCGKHSCSVDWGKATTCIINNGAMAWATGGHQGNHKC |
| 4 | KYYGNGVTCGKHSCSVDWGKATTCIINNGALAWATGGHQGNHKC |
| 5 | KYYGNGVTCGKHSCSVDWGKATTCIINNGALAWATGGHQGNHKC |
| 6 | KYYGNGVTAGKHSASVDWGKATTAIINNGAMAWATGGHQGNHKA |
| 7 | KRKKHRCRVYNNGMPTGMYRWC |
| 8 | KRKKHRCRVYNNGLPTGLYRWC |
| 9 | H₂N-PedM31L-CONH₂ |
| 10 | Ac-NH-PedM31L-CONH₂ |

To avoid the oxidation of the Met31 residue during the synthesis, antibacterial assays and conformational studies, a linear analog of pediocin PA-1 (4) containing a Leu31 residue was prepared as described above. Following purification as described above, pediocin PA-1 4 was obtained in 40% overall yield. Pediocin PA-1 4 was subsequently submitted to disulfide bond formation using NCS to afford pediocin PA-1 and analogue 5 (PA-1 M31L). A reduction protocol was obviated in view of the peptide not containing a Met31 residue.

To demonstrate the importance of the disulfide bonds to the antimicrobial activity of pediocin PA-1 and to maintain its bioactive conformation, a further pediocin PA-1 analog (6) was prepared as described above. Relative to pediocin PA-1 3, pediocin PA-1 6 comprises alanine residues Ala9, Ala14, Ala24 and Ala44. In essence, the Cys residues in PA-1 3 were substituted for Ala residues in PA-1 6.

The antimicrobial activity of pediocin PA-1 1, 2a, 3a-c and pediocin analogues 4 and 6 was assessed by determining the minimal inhibitory concentration against Listeria ivanovii HPB28, Listeria monocytogenes LSD530, Listeria monocytogenes ATCC 19111, Micrococcus luteus ATCC 10240 and Pediococcus acidilacti UL5. The observed respective minimal inhibitory concentrations are given in Table 4. Synthetic pediocin PA-1 3c showed strong activity with a low nanomolar MIC of 6.75 nM against L. ivanovii HPB28 and L. monocytogenes LSD530 respectively, and a nanomolar MIC of 13.5 nM against L. monocytogenes ATCC 19111. Compared to PA-1 3c, PA-1 3a and 3b, the latter two peptides having an incorrect disulfide bond pairing, showed a 2- to 4-fold decrease in activity against Listeria ivanovii HPB28, Listeria monocytogenes LSD530 and Listeria monocytogenes ATCC 19111 (MIC ranging from 13.5-27.0 nM). The enhanced antimicrobial activity of PA-1 3c relative to PA-1 3a and 3b, could at least in part be explained by PA-1 3c exhibiting an energetically more favorable conformation. Indeed, as per the observed mixture of 2a (7.5%), 2b (21.5%) and 2c (71.5%), there appears to be a thermodynamic equilibrium in the disulfide pairings favoring the conformation exhibited by PA-1 3c. As expected, a significant decrease of antimicrobial activity was observed for PA-1 2a comprising an oxidized Met31 residue. PA-1 2a exhibited an MIC of 1562 nM against Listeria ivanovii HPB28 and Listeria monocytogenes LSD530, and an MIC of 25000 nM against Listeria monocytogenes ATCC 19111. These results confirm that the oxidation of the Met31 residue is detrimental to the activity of PA-1.

TABLE 4

Minimal inhibitory concentrations (MIC) of synthetic Pediocin PA-1 3c and analogues for selected bacteria

| Strain | | | Minimal Inhibitory Concentration (nM) | | | | | | | |
|---|---|---|---|---|---|---|---|---|---|---|
| | 1 | 2c | 3a | 3b | 3c | 4 | 5 | 6 | 9 | 10 |
| Listeria ivanovii HPB28 | 6.8 | 1562 | 27.0 | 13.5 | 6.8 | 6.8 | 1.7 | N/A ¹ | N/A | N/A |
| Listeria monocytogenes LSD530 | 13.5 | 1562 | 27.0 | 13.5 | 6.8 | 13.5 | 6.8 | N/A | N/A | N/A |
| Listeria monocytogenes ATCC 19111 | 13.5 | 25000 | 27.0 | 13.5 | 13.5 | 13.5 | 13.5 | N/A | 90.0 | 1000 |
| Micrococcus luteus | N/A | N/A | N/A | N/A | N/A | N/A | N/A | N/A | N/A | N/A |
| P. acidilacti UL5 | N/A | N/A | N/A | N/A | N/A | N/A | N/A | N/A | N/A | N/A |

¹(-) No activity detected at concentrations up to 100 µM.

Surprisingly, linear pediocin PA-1 1 and linear analogue 4 showed similar antimicrobial activity in radial diffusion and microplate dilution assays. Interestingly, these results suggest that the disulfide bonds can be suitably formed in situ in the bioassay medium, without the help of chaperone-like proteins.[5] The observed increased antimicrobial activity of linear pediocin PA-1 1 relative to PA-1 3c, could at least in part be explained by the presence of the free Cys residues acting as oxidant scavengers, preventing the oxidation of the Met31 during disulfide bond formation. The observed antimicrobial activity for linear analog 4 is in agreement with the MIC values previously reported.[22] Regarding PA-1 3c, it is possible that a small amount of Met31 gets oxidized during the assay, thus decreasing its antimicrobial activity. No antimicrobial activity was observed for linear analog 6, indicative of the disulfide bonds being essential for the activity of pediocin PA-1. Even though antimicrobial activity of pediocin PA-1 against Micrococcus luteus has recently been reported[25], no activity against M. luteus ATCC 10240 could be observed for synthetic pediocin PA-1 3c, PA-1 3a, 3b and linear analogues 4 and 6 at up to 100 µM. It is surmised that this could be the result of low agitation of the cells which need to be oxygenated or again fast methionine oxidation. Finally, none of the synthesized peptides showed antimicrobial activity against P. acidilacti UL5.

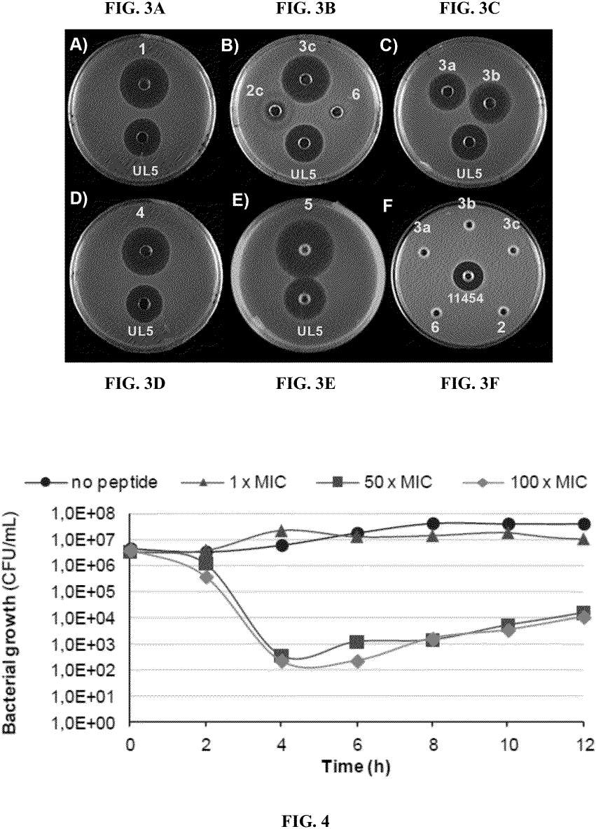

In order to confirm the results as obtained in the MIC assay, the antibacterial activity of the synthetized peptides was further assessed by radial diffusion assays against L. monocytogenes LSD530 and compared to native pediocin PA-1 produced by P. acidilacti UL5 (FIG. 3). In agar plate assays against L. monocytogenes LSD530, the presence of pediocin PA-1 in the supernatant of P. acidilacti UL5 was confirmed by the observed antimicrobial activity with an inhibition diameter of 24 mm (FIGS. 3A-D). As expected, synthetic pediocin PA-1 3c showed a substantial inhibition diameter of 31 mm while poor and no activity was observed for analogs 2c and 6, respectively (FIG. 3A). As previously observed with the MIC assay, reduced activity was observed for PA-1 3a and PA-1 3b exhibiting inhibition diameters of 24 and 27 mm, respectively (FIG. 3B). Linear pediocin PA-1 1 and linear analogue 4 exhibited excellent antimicrobial activity as per the observed inhibition diameters of 32 mm (FIGS. 3C and 3D). These results again suggest in situ disulfide bond formation for PA-1 1 and linear analogue 4.[5] Finally, none of the synthetic peptides 2c, 3a-c, and 6 shows antimicrobial activity against P. acidilacti UL5 (FIG. 3F)

To corroborate the possibility of in situ disulfide bond formation, bactofencin A (BAC221) linear analogs 7 and 8 were prepared as described above and evaluated in the radial diffusion assay (FIG. 3E). Bactofencin A, a bacteriocin isolated from a porcine intestinal bacteria Lactobacillus salivarius DPC6502, and consisting of a short 22 residue, defensin-like sequence, including a disulfide bridge and a methionine residue, is known to be active against both L. monocytogenes and Staphylococcus aureus.[24] Bactofencin A linear analogs 7 and 8 exhibited inhibition diameters of 9 and 11 mm, respectively against L. monocytogenes ATCC 19111. These results are in accordance with previously reported inhibition diameters for native bactofencin A against the aforementioned strains. These results again support in situ disulfide bond formation and further suggest that the Met residues mostly function as hydrophobic residues in the class II bacteriocin mechanism of action. Moreover, the Met18 residue of analogue 7 can be replaced with a Leu18 (analogue 8) enhancing the stability of the peptide.

Synthetic bactofencin A linear analogs 7 and 8 exhibited low micromolar MIC values of 5.8 µM and 2.8 µM against S. aureus ATCC 6538 and L. monocytogenes ATCC 19111, respectively (Table 5).

TABLE 5

Minimal inhibitory concentrations (MIC) of synthetic Bactofencin A linear analogs 7 and 8

| Strain | Minimal Inhibitory Concentration (µM)* | |
|---|---|---|
| | 7 | 8 |
| Listeria monocytogenes ATCC 19111 | 8.02 | 4.06 |
| Staphylococcus aureus ATCC 6538 | 4.01 | 2.00 |

*Initial Concentration 0.25 mg/mL

To evaluate the in situ disulfide bond formation in a biological matrix, the antimicrobial activity of different concentrations of linear pediocin PA-1 analog 4 against L. monocytogenes ATCC19111 at 30° C. was assessed in skim milk. The average bacterial growth (CFU/mL) for 12 h is shown (FIG. 4).

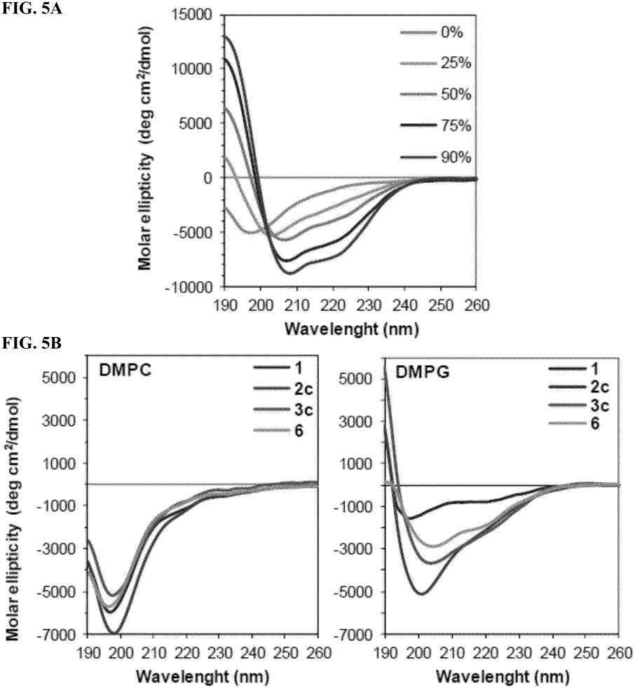

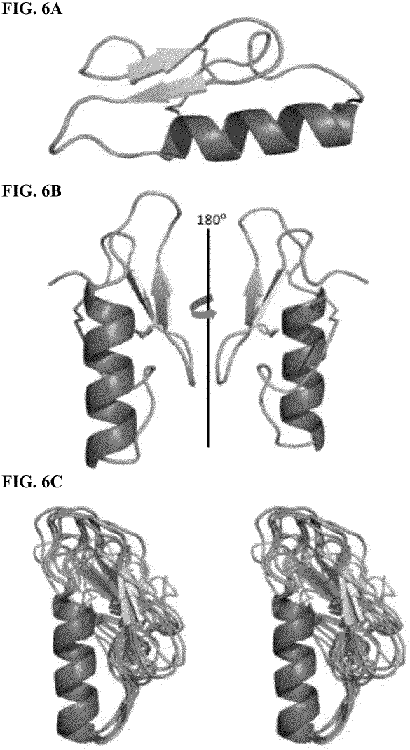

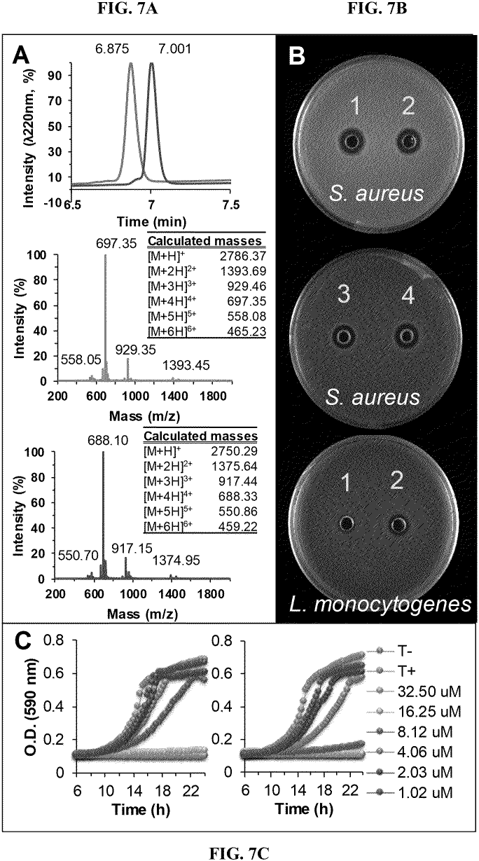

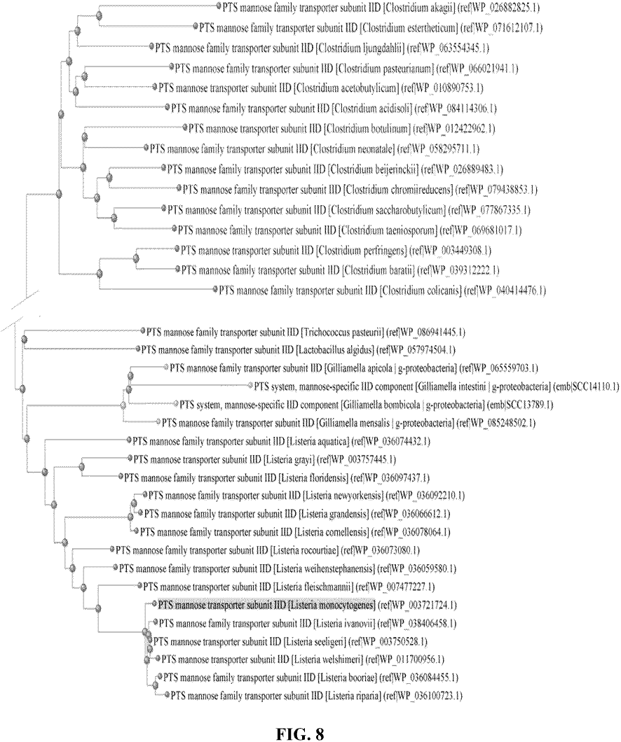

Circular dichroism experiments were performed (FIG. 5) in order to determine the optimal conditions for the ¹H NMR spectroscopy studies of the peptides. A solvent mixture composed of trifluoroethanol (TFE-d2) and H₂O (50/50) was used for the ¹H NMR structure analyses. The pH of the solution was maintained under a value of 3 in order to maintain the disulfide bridges in the appropriate pairings. Moreover, Pediocin PA-1 M31L 5 was used in order to prevent oxidation and structural changes during the acquisition times. Moreover, leucine is well known to mimic the electrostatic surface of the methionine. One-dimensional ¹H NMR and two-dimensional homonuclear ¹H-¹H total correlation spectroscopy (TOCSY) and nuclear Overhauser effect spectroscopy (NOESY) data sets were acquired. FIG. 6 illustrates the lowest relative energy structure (α-helix red, β-sheet yellow, loops green and disulfide bridge orange) for synthetic pediocin PA-1 analog 5 as determined by ¹H NMR spectroscopy.

In an aspect, the present disclosure relates to a process for the linear synthesis of gram-positive class IId bacteriocins and compositions and uses thereof. In an embodiment, the present disclosure relates to the linear synthesis of bactofencin A and compositions and uses thereof. In a further embodiment of the present disclosure, the synthesis of gram-positive class IId bacteriocins comprises the use of linear solid phase peptide synthesis. In yet a further embodiment of the present disclosure, the process for the synthesis of bactofencin A comprises the use of linear solid phase peptide synthesis. In yet a further embodiment of the present disclosure, the various peptides and analogues thereof, were prepared by solid phase peptide synthesis using the Fmoc-Nα/t-Bu strategy on a HMPB-ChemMatrix® solid support or Rink-ChemMatrix® solid support.

In an aspect, the present disclosure relates to the linear solid phase peptide synthesis of bactofencin A and variants thereof. It is to be understood that all process/method steps described herein are to be conducted under conditions sufficient to provide the desired end product (e.g. a gram-positive class IId bacteriocin). A person skilled in the art would understand that all processing conditions, including, for example, processing time, processing temperature, and whether or not the process should be performed under an anhydrous or inert atmosphere, can be varied to optimize the yield of the desired product and it is within their skill to do so.

It will be apparent to one skilled in the art, that in the course of peptide synthesis, it may be necessary to protect certain side chains of the amino acids to prevent unwanted side reactions. For example, it may be necessary to protect the hydroxyl group on the side chain of tyrosine, serine, or threonine in order to prevent these groups from interfering with the desired reactions. This is a common problem in peptide synthesis and many procedures are available for protecting the functional groups on the side chains of the amino acids. Such procedures for protecting various functional groups are known to one skilled in the art and are described in the treatise entitled “PEPTIDES: CHEMISTRY AND BIOLOGY”, Norbert Sewald and Hans-Dieter Jakubke, 2^{nd} Edition, Wiley-VCH Verlag GmbH & Co., Weinheim, 2009, and the reference book Protective Groups in Organic Synthesis, 3^{rd} Edition, by T.W. Green and P.G.M. Wuts, John Wiley and Sons, New York, 2002, the contents of both being incorporated herein by reference.

Straight forward Fmoc-Nα/t-Bu solid phase peptide synthesis (SPPS) on a HMPB and Rink-ChemMatrix®, Rink AM and 2-Chlorotrityl polystyrene resin provided for the linear synthesis of Bactofencin A as well as variants thereof (Table 6).

TABLE 6

Bactofencin A and variants thereof and their activity against S. aureus ATCC 6538 cultivated in MH broth

| | | | | |
|---|---|---|---|---|
| Peptide | Sequence | DOI¹ (mm) | MIC₅₀ (µM) | MIC_{%}² (%) |
| 1 | KRKKHRCRVYNNGMPTGMYRWC | 15 | 4.01 | 25 |
| 2 | KRKKHRCRVYNNGLPTGLYRWC | 15 | 2.00 | 50 |
| 3 | KRKKHRCRVYNNGLPTGLYRWC-NH₂ | 13 | 1.02 | 100 |
| 4 | Ac-KRKKHRCRVYNNGLPTGLYRWC-NH₂ | 12 | 2.03 | 50 |
| 5 | ---KHRCRVYNNGLPTGLYRWC-NH₂ | 15 | 4.78 | 25 |
| 6 | ----HRCRVYNNGLPTGLYRWC-NH₂ | 11 | 10.1 | 12.5 |
| 7 | -----RCRVYNNGLPTGLYRWC-NH₂ | 10 | 21.6 | 6.25 |
| 8 | ------CRVYNNGLPTGLYRWC-NH₂ | n.a.³ | 187 | 0.78 |
| 9 | KRKKHRCRVFNNGLPTGLYRWC-NH₂ | 10 | 4.08 | 25 |
| 10 | KRKKHRCRVWNNGLPTGLYRWC-NH₂ | 9 | 8.05 | 12.5 |
| 11 | KRKKHRCRVYNNGLPTGLFRWC-NH₂ | 12 | 1.02 | 100 |
| 12 | KRKKHRCRVYNNGLPTGLSRWC-NH₂ | 12 | 2.09 | 50 |
| 13 | KRKKHRCRVYNNGLPTGLWRWC-NH₂ | 12 | 2.01 | 50 |
| K1A | ARKKHRCRVYNNGLPTGLYRWC-NH₂ | 13 | 1.02 | 100 |
| R2A | KAKKHRCRVYNNGLPTGLYRWC-NH₂ | 14 | 2.03 | 50 |
| K3A | KRAKHRCRVYNNGLPTGLYRWC-NH₂ | 12 | 1.02 | 100 |
| K4A | KRKAHRCRVYNNGLPTGLYRWC-NH₂ | 12 | 4.06 | 25 |
| H5A | KRKKARCRVYNNGLPTGLYRWC-NH₂ | 11 | 2.03 | 50 |
| R6A | KRKKHACRVYNNGLPTGLYRWC-NH₂ | 12 | 2.03 | 50 |
| C7A | KRKKHRARVYNNGLPTGLTRWC-NH₂ | 8 | 16.25 | 6.25 |
| R8A | KRKKHRCAVYNNGLPTGLYRWC-NH₂ | 11 | 4.06 | 25 |
| V9A | KRKKHRCRAYNNGLPTGLYRWC-NH₂ | 9 | 32.50 | 3.13 |
| Y10A | KRKKHRCRVANNGLPTGLYRWC-NH₂ | 8 | 32.50 | 3.13 |
| N11A | KRKKHRCRVYANGLPTGLYRWC-NH₂ | 8 | 16.25 | 6.25 |
| N12A | KRKKHRCRVYNAGLPTGLYRWC-NH₂ | 8 | 8.12 | 12.5 |
| G13A | KRKKHRCRVYNNALPTGLYRWC-NH₂ | 8 | 32.50 | 3.13 |
| L14A | KRKKHRCRVYNNGAPTGLYRWC-NH₂ | 9 | 16.25 | 6.25 |
| P15A | KRKKHRCRVYNNGLATGLYRWC-NH₂ | 10 | 4.06 | 25 |
| T16A | KRKKHRCRVYNNGLPAGLYRWC-NH₂ | 8 | 64.99 | 1.56 |
| G17A | KRKKHRCRVYNNGLPTALYRWC-NH₂ | 9 | 8.12 | 12.5 |
| L18A | KRKKHRCRVYNNGLPTGAYRWC-NH₂ | 11 | 4.06 | 25 |
| Y19A | KRKKHRCRVYNNGLPTGLARWC-NH₂ | 12 | 64.99 | 1.56 |
| R20A | KRKKHRCRVYNNGLPTGLYAWC-NH₂ | 12 | 32.50 | 3.13 |
| W21A | KRKKHRCRVYNNGLPTGLYRAC-NH₂ | 11 | 4.06 | 25 |
| C22A | KRKKHRCRVYNNGLPTGLYRWA-NH₂ | n.a. | n.a. | n.a. |

¹DOI = Diameter of inhibition; ²MIC_{%} = % of inhibition is based on analog 3 used for ala-scan; ³n.a. = no activity observed at tested concentrations of 1 mg/mL.

Leucine for methionine substitution resulted in variants having increased activity. Indeed, enhanced activity was observed for peptide 2 relative to peptide 1 (2.00 µM versus 4.06 µM respectively) against S. aureus. Substituting the HMPB linker for the Rink linker resulted in the isolation of peptides (following cleavage from the solid state resin) having an amidated C-terminal. In an embodiment of the present disclosure, bactofencin A-based variants were prepared comprising an acetylated N-terminal (e.g. peptide 4). Peptide 3 comprising an amidated C-terminal was shown to exhibit enhanced activity relative to peptides 1 and 2 respectively against S. aureus. However, peptide 4, comprising both an amidated C-terminal and an acetylated N-terminal, was shown to exhibit an activity against S. aureus similar to peptide 2. Substitution of the HMPB-ChemMatrix for the 2-TCP resin, resulted in higher yields of isolated peptides (25.8% to 62.6% respectively) following purification. Peptide 2, obtained by replacement of the methionine residues for leucine residues, was shown to exhibit better activity against both S. aureus and L. monocytogenes, suggesting that the methionine residues can be replaced in order to enhance the oxidative resistance of the peptide. Cysteine replacement resulted in 6.25% activity for peptide C7A and a complete loss of activity for peptide C22A. Various alanine substitutions, as for peptides K1A-C22A were also performed. The intramolecular disulfide bond seems to be essential for the activity. Indeed, substitution of the cysteine residues at positions 7 and 22 respectively resulted in an important loss of activity for C7A to complete inactivation for C22A respectively. The loss of activity observed for peptides wherein one or both of the cysteine residues have been substituted further emphasizes the importance of in situ disulfide bond formation in the activity of the peptides. Moreover, in situ disulfide bond formation is further corroborated by the activity observed for the use of linear peptides comprising cysteine residues in a biological medium.

### EXPERIMENTAL

A number of examples are provided herein below illustrating the process for the linear synthesis of gram-positive class II bacteriocins and compositions and uses thereof. In accordance with various embodiments of the present disclosure, a number of examples are provided hereinbelow illustrating the linear solid support peptide synthesis of pediocin PA-1 and compositions and uses thereof. In accordance with various embodiments of the present disclosure, a number of examples are provided hereinbelow illustrating the linear solid support peptide synthesis of bactofencin A and compositions and uses thereof. The following non-limiting examples are illustrative of the present disclosure.

### Example 1: Materials

All reagents and solvents were purchased from commercial suppliers and used without further purification. Fmoc amino acid derivatives, coupling reagents (e.g. 2-(7-aza-1H-benzotriazole-1-yl)-1,1,3,3-tetramethyluronium hexafluorophosphate (HATU), 2-(1H-benzotriazol-1-yl)-1,1,3,3-tetramethyluronium hexafluorophosphate (HBTU), 2-(6-chloro-1H-benzotriazole-1-yl)-1,1,3,3-tetramethylammonium hexafluorophosphate (HCTU) and 1-(mesitylene-2-sulfonyl)-3-nitro-1,2,4-triazole (MSNT) were purchased from Matrix innovation (Quebec, QC, Canada). Aminomethyl-ChemMatrix® resin (0.69 mmol/g) was purchased from PCAS Biomatrix Inc. (St-Jean-sur-Richelieu, QC, Canada). Pseudoproline derivatives were purchased from Gyros Protein Technologies (Tucson, AZ, USA). Linker 4-(4-hydroxymethyl-3-methoxyphenoxy)-butyric acid (HMPBA) was purchased from Chem-Impex (Wood Dale, IL, USA). Other reagents and solvents were purchased from Sigma-Aldrich.

### Example 2: Analytical Analyses

LC/MS analyses were conducted on a Shimadzu Prominence LCMS-2020 system equipped with an ElectroSpray Ionization (ESI) probe and using a Kinetex® column (4.6 mm x 100 mm, 2.6 µm XB-C18, 100 Å, 1.8 mL/min) and a 10.5 min. gradient from water (0.1% HCOOH) and CH₃CN (0.1% HCOOH) (CH₃CN 10-100%) and detection at 220 nm and 254 nm. High Resolution Mass Spectrometry (HRMS) was performed on a Waters Synapt G2-Si (Quadrupole/TOF) equipped with a Waters UPLC binary pump and an FTN (Flow-Through Needle) injector. The mass spectrometer was operated in High resolution mode; calibration was performed using a sodium formate solution; and lock-mass correction was performed using a Leucine-enkephalin solution (Waters). Matrix-Assisted Laser Desorption Ionization Time-of-Flight (MALDI-TOF) mass spectrometry was performed using a AB SCIEX 4800 Plus MALDI-TOF/TOF® instrument equipped with an alpha-cyano-4-hydroxycinnamic acid matrix. The spectra were acquired using the 4000 Series Explorer Software (Ab Sciex, v 3.2.3). The PEAKS Studio software (Bioinformatics Solutions, v.7.0) was used for spectra analysis and DENOVO sequencing.

### Example 3: Peptide Synthesis

Peptides were synthesized by standard Fmoc solid-phase synthesis with appropriate orthogonal protection and resin linker strategies using a Prelude automated peptide synthesizer from Gyros Protein Technologies (Tucson, AZ, USA) and an HMPB-ChemMatrix® resin. The HMPB-ChemMatrix resin was prepared by swelling aminomethyl-ChemMatrix in DMF for 1 h followed by the addition of 4-(4-hydroxymethyl-3-methoxyphenoxy)-butyric acid (HMPBA) (3 equiv.), HBTU (3 equiv.), HOBt (3 equiv.) and N-methylmorpholine (NMM) (6 equiv.) respectively. After stirring the mixture for 3 h, the resin was washed with DMF (dimethylformamide) (5x) and DCM (dichloromethane) (5x) and dried under vacuum. The C-terminal amino acid was subsequently attached by dissolving Fmoc-Cys(Trt)-OH (5 equiv.) in DCM with a minimum amount of anhydrous THF, and adding the resulting solution to the previously prepared resin swollen in DCM. MSNT (1-(mesitylene-2-sulfonyl)-3-nitro-1,2,4-triazole) (5 equiv.) and N-methylimidazole (3.75 equiv.) were dissolved in DCM and incubated with the resin for 1 h under agitation. After washing the resin with DCM (5x) and DMF (5x), peptide elongation was carried out by standard Fmoc solid phase peptide synthesis: the Fmoc protecting group was removed from the resin by two 10 min treatments with 20 % piperidine in DMF (v/v); and amino acid couplings were performed using Fmoc-Xaa-OH (3 equiv.), HCTU (3 equiv.) and NMM (12 equiv.) in DMF (2x 30 min). Cleavage, side chain deprotection, and pseudoproline ring reopening were respectively achieved by treatment with TFA/TIPS (triisopropylsilane)/H₂O/phenol (90:5:2.5:2.5) over a period of 1 h. After precipitation in cold ether, a second side chain deprotection treatment using TFA/TIPS/H₂O (95:2.5:2.5) was performed to ensure complete deprotection and pseudoproline ring opening. The resulting peptide was subsequently precipitated using cold diethyl ether, washed twice with diethyl ether and dried under vacuum. Finally, the peptide was purified by RP-HPLC (Reversed phase HPLC) using a Shimadzu Prominence instrument equipped with a Phenomenex Kinetex® EVO C18 column (250 mm × 21.2 mm, 5.0 µm, 300 Å) and using 0.1% AcOH/H₂O (solvent A) and 0.1% AcOH/CH₃CN (solvent B) with a linear gradient of 5% to 50% (for solvent B) over a period of 20 min at 14 ml/min and UV detection at 220 and 254 nm respectively. The collected fractions were subsequently freeze-dried to afford the desired peptide as a white powder.

### Example 4: Disulfide Bond Formation

The purified linear peptide was dissolved in CH₃CN/H₂O (1:1) at a concentration of 1 mg/mL and cyclized by the addition of NCS (N-chlorosuccinimide) (4 equiv. or 2 equiv. per disulfide bond). After stirring for 30 min, the cyclized peptide product was freeze-dried and purified by RP-HPLC as described hereinabove.

### Example 5: Selective Methionine Reduction

Selective methionine sulfoxide reduction (oxidized Met) was carried out by treating the peptide at a concentration of 1 mg/mL with TBAB (tetrabutylammonium bromide) (30 equiv.) and TFA/β-mercaptoethanol/anisole (95:2.5:2.5) over a period of 5 min while at room temperature, followed by precipitation and washing using cold diethyl ether. The resulting peptide product was subsequently characterized by RP-HPLC and matrix-assisted laser desorption ionization time-of-flight mass spectrometry (MALDI-TOF).

### Example 6: Antimicrobial Assays

The antibacterial activity of the synthetic peptides in accordance with an embodiment of the present disclosure, was assessed by radial diffusion assays against L. monocytogenes LSD530 from the Canadian Food Inspection Agency (Laboratory Services Division, Ottawa, ON, Canada), L. monocytogenes ATCC 19111 and M. luteus 10240 from the American Type Culture Collection (Rockville, MD, USA), P. acidilacti UL5 from the STELA Dairy Research Center culture collection (Université Laval, QC, Canada), and Listeria ivanovii HPB28 (Health Protection Branch, Health and Welfare, Ottawa, ON, Canada). A sample (80 µL) containing 1 mg/mL of purified peptide was applied into the hole of a MRS (Oxoid, Nepean, ON, Canada) or TSBYE soft agar (0.75% w/v) overlay seeded with the producer strain P. acidilacti and the indicator strain of L. monocytogenes LSD530. The petri plates (100 × 15 mm) (VWR, Radnor, PA, USA) were incubated at 35° C. for 18 h and the antibacterial activity was observed as a halo of inhibition formed in the bacterial carpet around the sample of the indicator strain. Nisin supernatant was obtained from Lactococcus lactis subsp. lactis ATCC 11454 in MRS broth at 30° C. after 18 h. All strains were reactivated from 20% glycerol stock at -80° C. and sub-cultured at least three times at 24 h intervals before use. Pictures were taken using the ChemiDoc® XRS imaging system (Bio-Rad, Hercules, CA, USA).

The minimal inhibitory concentrations (MIC) of the synthetic peptides were determined using 96-well Falcon® polystyrene micro-assay plates (Corning, NY, USA). Micro-plates loaded with twofold serial dilutions of each peptide (starting at 250 µM) in tryptic soy broth (Difco Laboratories, Sparks, MD, USA) supplemented with 0.6% yeast extract (w/v) were seeded with log-phase culture of target strain diluted in TSBYE to 0.5-1.0 × 10⁶ cfu mL⁻¹ (approximately 1 × 10⁴ cfu per well). The micro-plates were then incubated at 30° C. for 18 h and the absorbance at 595 nm was measured hourly using an Infinite® F200 PRO photometer (Tecan US, Inc., Durham, NC, USA). The MIC values were expressed in µM and correspond to the lowest concentration that inhibited the growth of the target organism after 18 h. The MIC values are reported as means of two independent experiments performed in duplicate.

The skim milk experiment was done in a preparation of 12% sterilized skim milk. L. monocytogenes ATCC 19111 was inoculated at approximately 10⁶ cfu/mL. Serial dilutions were performed and the cfus counted after 2, 4, 6, 8, 10 12, 24 and 48 h using 20 µL for each replicate from each dilution on TSBYE agar plate (1.25% w/v) and incubated for 24 h.

### Example 7: Circular Dichroism (CD)

Peptides were dissolved in 0.1% TFA/H₂O (1 mg/mL) and diluted to 0.1 mM in aqueous TFE solutions (0, 25, 50, 75 or 90% TFE in H₂O). For the study in phospholipid vesicles, a lipid/peptide ratio of 100:1 was used. DMPC (dimyristoylphophatidylcholine) or DMPG (dimyristoylphophatidylglycerol) was dissolved in MeOH and the mixture dried with a stream of nitrogen. The peptides were subsequently dissolved in phosphate buffer (20 mM, pH 7.4) (1 mg/mL) and added to the dried phospholipid films. Finally, the micelles were sonicated for 5 min or until a clear solution was obtained. CD measurements of the peptides in aqueous TFE solutions and in phospholipid vesicles were performed using a Jasco J-815 Circular Dichroism Spectropolarimeter (Aviv Instruments, Lakewood, NJ, USA). The spectra were recorded at 25° C. in the 190-260 nm wavelength range, at 0.1 nm intervals, in a cuvette with a 0.1 mm path length. For each spectrum, ten scans were averaged and smoothed using the J720/98 system program (Version 120C). CD data were expressed as mean residue molar ellipticity [θ] expressed in deg cm² dmol⁻¹, plotted against wavelength (nm) and analyzed using the CONTIN algorithm included in the CDPro analysis software.

### Example 8: NMR Spectroscopy

Samples were prepared using 2 mg of Pediocin PA-1, 3c, and ped[M31L] 5 dissolved in a solution of H₂O with 0.1% TFA (300 µL) and TFE-d2 98% (300 µL) in a 3 mm Wilmad NMR tube obtained from Rototec-Spintec. Experiments were performed using a Bruker Avance 600 MHz spectrometer equipped with a cryoprobe. The temperature effect on the structure was surveyed by recording ¹H NMR spectra at variable temperatures (288, 298, 303, 308, 313 °K) and water suppression using sculpting with gradients. For sequential assignments, TOCSY and NOESY experiments were performed in phase-sensitive mode. TOCSY and NOESY spectra were recorded with mixing times of 80 ms and 300 ms respectively at 313 °K and for 16 and 72 scans respectively. Water suppression was achieved using excitation sculpting. All spectra were processed with Bruker TOPSPIN® 3.5 software.

### Example 9: In Situ Disulfide Bond Formation

Incorrect disulfide bond pairings such as in analogs 3a and 3b reduced inhibitory activity. These results suggest that the disulfide bonds may exist in a dynamic equilibrium that allows for some remodeling in the culture medium to produce a small quantity of the bioactive conformation. Surprisingly, equivalent antimicrobial activities were observed for linear analogs 1 and 4 in radial diffusion and microplate dilution assays (Table 4). These results support the in situ disulfide bond formation hypothesis proposing that suitable disulfide bonds can be formed in the bioassay medium without the help of chaperone-like proteins.[5] Based on the disulfide bond pairing ratios of 2a (7.5%), 2b (21.5%) and 2c (71.0%) obtained after cyclization with NCS and the similar antimicrobial activities, a similar equilibrium could be reached in culture medium containing linear analogues 1 and 4 but also native pediocin PA-1 3c. Substitution of all four cysteine residues with alanine showed that the disulfide bonds are essential for pediocin PA-1 activity, since linear analog 6 was inactive which lends further support for in situ disulfide bond of the linear analogues.

### Example 10: Bacterial Targets of Pediocin Pa-1 M31l

A phylogenetic analysis was performed to determiner if other bacteria contained in the mannose phosphotransferase system (Man-PTS IICD protein) were targeted by pediocin PA-1 M31L (5) (FIG. 8). It was determined that Clostridium perfringens and Clostridium botulinum, which are important foodborne pathogens, could be advantageously inhibited by pediocin PA-1 M31L (5). Indeed, against Clostridium perfrigens pediocin PA-1 M31L’s MIC is between 27.8-75.7 nM. In addition to targeting Gram-positive bacteria, the possibility for Pediocin to also target Gram-negative bacteria, if combined with other treatments such as heating, has been reported. Indeed, some bacteria such as Salmonella typhimurium, Escherichia coli, Serratia liquefaciens and Pseudomonas fluorescens are sensitive to pediocin PA-1 M31L (5) when their membrane has been previously damaged.[8, 26, 27]

### Example 11: Efficacy Testing of Food Processing Aids Against Listeria Monocytogenes

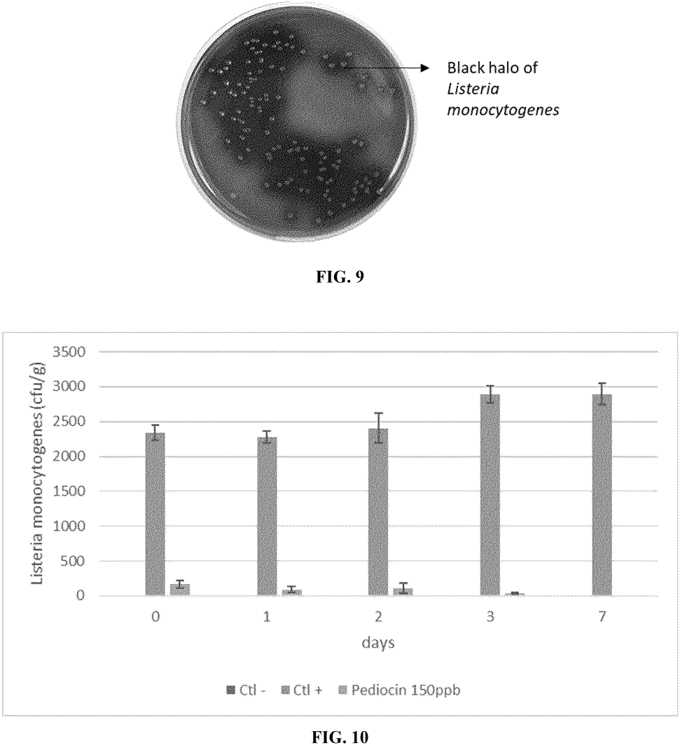

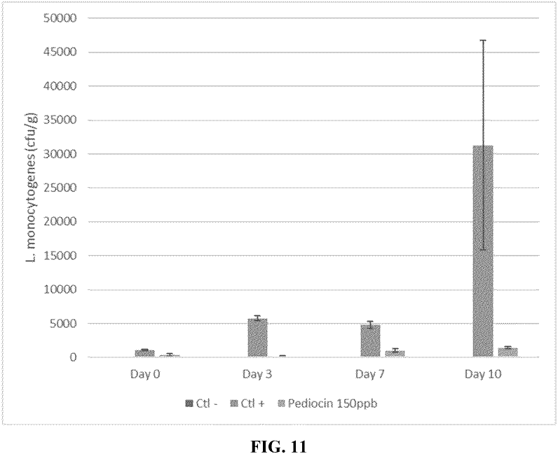

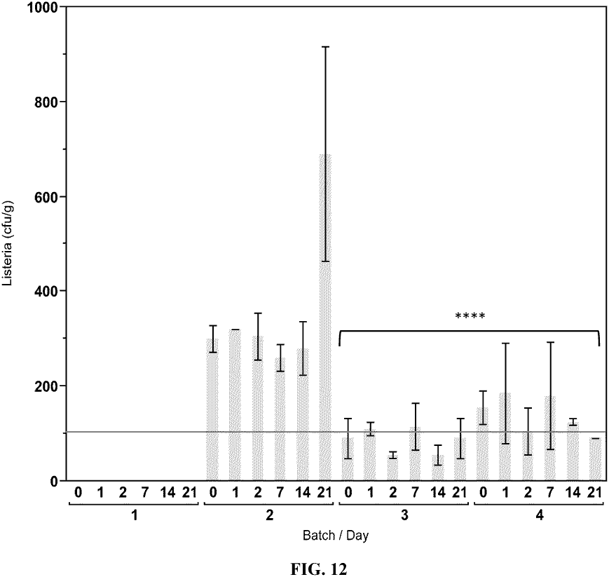

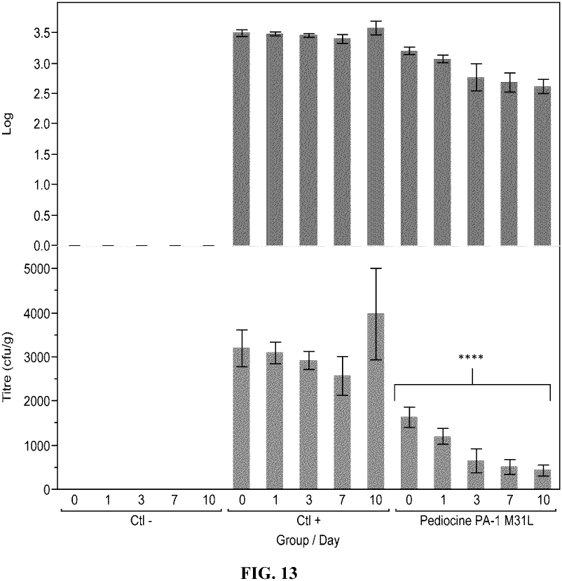

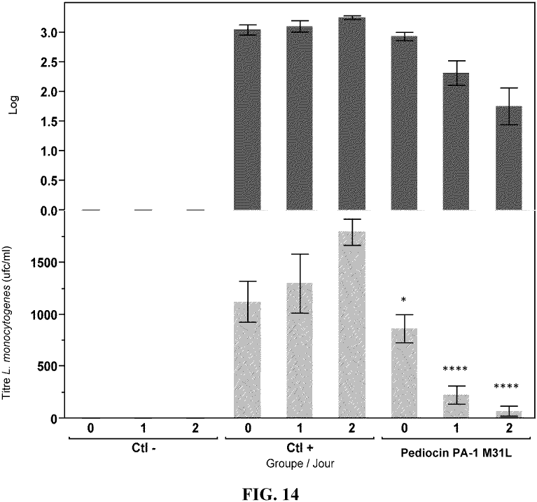

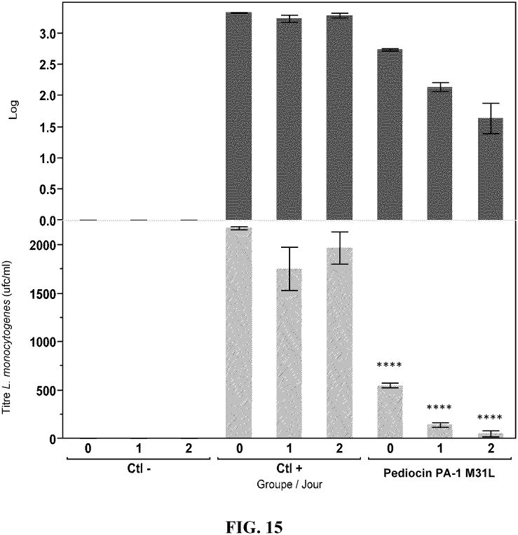

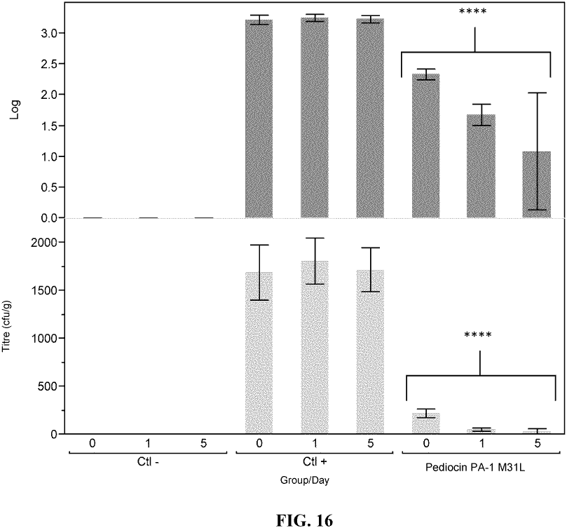

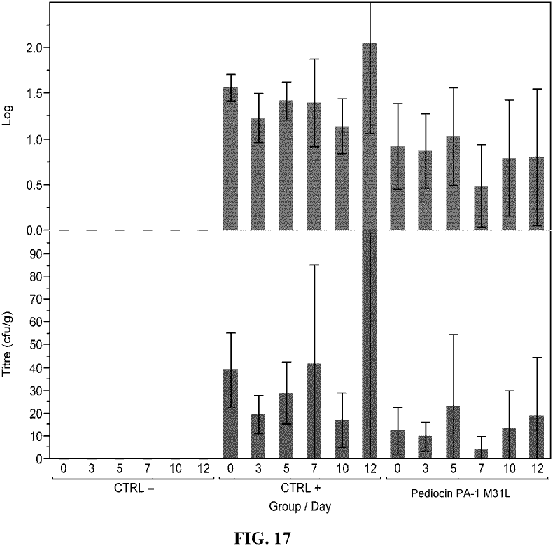

The use of food processing aids as potential antimicrobial agents against Listeria monocytogenes was determined. To test the efficiency of a processing aid against Listeria monocytogenes, the Health Canada protocol for performing challenge tests was used for different types of matrices (Listeria monocytogenes Challenge Testing of RTE food, 2010). It also meets all the parameters of a combined growth and inactivation study according to the guidelines of the Parameters for Determining Inoculated Pack/Challenge Study Protocol by the National Advisory Committee on Microbiological Criteria for Food. [28] In fact, a cocktail of three different serotypes (½a, ½b, and 4b) of Listeria monocytogenes were inoculated on the different food matrices. At least one of the strains needed to be specifically adapted for the type of food and one serotype should source from an outbreak (Table 7). In parallel, to verify the morphological identification of the inoculated bacteria on the matrices, the experiments were performed according to the official method of Health Canada (Listeria monocytogenes Challenge Testing of RTE food, 2010). Listeria monocytogenes is easy to identify by the formation of a black halo on PALCAM (FIG. 9).

TABLE 7

Strains and serotype inoculum used for each food matrix to perform the challenge tests

| Listeria monocytogenes | Serotype | Company | Isolate source | Origin from an outbreak or not |
|---|---|---|---|---|
| J1-108 | 4b | IAFNS | Sauerkraut | yes¹ |
| L3-0051 | ½b | IAFNS | Fish | no |
| F2-0237 | ½a | IAFNS | Fish | no |
| FSL-G1-200 | ½a | IAFNS | Avocado pulp | no |
| 10494 | ½b | DSMZ | Poultry | no |
| 517772 | ½a | ATCC | Dairy product | no |

¹Human epidemic, coleslaw, Halifax, 1981; Cornell University Food Safety Lab.
ATCC: American type culture collection, DSMZ: German collection of microorganisms and cells cultures, IAFNS: Advanced Food and Nutrition Sciences at Cornell University

### Example 12: Challenge Test - Anti-Listeria Activity

To demonstrate the efficacy of Pediocin PA-1 M31L (5) on various types of matrices, challenge tests were performed according to Health Canada guidelines Listeria monocytogenes Challenge Testing of RTE food, 2010. The food matrices used were obtained from partnerships with food processors or bought in grocery stores. Two different concentrations of pediocin PA-1 M31L (5) (50 and 150 ng/g (50 and 150 ppb)) were tested to determine which concentration is more efficient in accordance with the degradation tests on food products. In summary, challenge tests were performed for 4 to 5 different analysis times (4 h, 24 h, 48 h, 1X shelf life, and 1.5X shelf life) according to the respective shelf life of the food. This allows for a determination if there is an immediate effect of pediocin PA-1 M31L (5) on Listeria monocytogenes and if this effect is maintained through time. This experiment also allows to measure the Log CFU/g reduction of Listeria monocytogenes.

### Example 13: Design of the Challenge Study

This experiment is a proof-of-concept to demonstrate the antimicrobial activity of pediocin PA-1 M31L (5) against the foodborne pathogen Listeria monocytogenes in a variety of foods (Table 9). All matrices were divided into four groups, representing different categories of food products. Targeted microorganisms: Each group was inoculated with a cocktail composed of three strains of Listeria monocytogenes specific for the group (Table 8). All the cocktails included a strain isolated from a listeriosis outbreak (strain 108) and a strain isolated from food in the group. All groups include three different serotypes (½a, ½b, and 4b).

TABLE 8

Strains of Listeria monocytogenes used in this study for each group

| Group 1: Fish and Seafood | Group 2: Poultry and Meat |
|---|---|
| 108 (Serotype 4b) | 108 (Serotype 4b) |
| 0051 (Serotype ½b) | DSM 19094 (Serotype ½a) |
| 237 (Serotype ½a) | 0051 (Serotype ½b) |
| Group 3: Fruit and vegetables | Group 4: Cheese and other dairy products |
| 108 (Serotype 4b) | 108 (Serotype 4b) |
| 0051 (Serotype ½b) | 0051 (Serotype ½b) |
| G1-02 (Serotype ½a) | ATCC 51772 (Serotype ½a) |

Batch division: Each experiment is divided into four batches, each sample of 25 g ± 0.5 g is analyzed in duplicate at each time point of the study. Batch 1 is the negative control (the unmodified food matrix). Batch 2 is the positive control (the food matrix inoculated with 10³ or 10⁵ cfu/g of Listeria monocytogenes). Batch 3 was inoculated at the same level of batch 2 but subsequently treated with 150 ng/g of pediocin PA-1 M31L (5). Batch 4 was inoculated at the same level of batch 2 but subsequently treated with 50 ng/g of pediocin PA-1 M31L (5).

Food preparation: Beef, pork, poultry, fresh salmon, and cheddar cheese curds were diced in order to increase the surface contact of the food, and therefore simulating the worst-case scenario of L. monocytogenes contamination. Shrimp and smoked salmon were kept intact (or cut into smaller slices to fit the 25 g restrain) because the surface contact ratio of these matrices is already high. Mixed vegetables were bought whole (not pre-cleaned), peeled (only for carrots) and cut manually to mimic a ready-to-eat process.

Inoculation: Strains were grown aerobically in TSBYE at 37° C. for 24 h and sub-cultured at 1% in the same conditions for 18 h. Each strain of the group is then diluted to reach a contamination level of 10³ to 10⁵ cfu/g of food (inoculation level is food dependant, see Table 9). All the strains of the group were pooled together to form the inoculation cocktail. All the samples of batch 2 to 4 were inoculated with 150 µl of the cocktail and spread evenly in the sample. Selected results are illustrated in Tables 10-15. Pediocin treatment: Batch 3 and 4 were respectively treated with 150 ng/g of Pediocin PA-1 M31L (5) and 50 ng/g of Pediocin. 1 ml ± 0.1 ml of the pediocin solution was sprayed onto the samples and gently mixed manually to cover the surface of the food to be tested.

Storage conditions: Each food tested was stored accordingly to the original conditions (see Table 9).

Sampling plan: Each food was analyzed on the day of the inoculation/treatment (4 h), at 24 h and 48 h. Samples were then analyzed at the expiration date and 1.5x the due date (see Table 9).

Enumeration method: MFLP-74 - Enumeration of Listeria monocytogenes in food, from the Compendium of Analytical Methods, Volume 3 of Health Canada. PALCAM was used as the plating media.

Statistical analysis method: The figures were made with the JMP 10.0.0 program and statistical analysis was performed by comparing the batches together for each day separately with an ANOVA and using “Student’s t” variable to compare the means. P-values on the graphs display only the differences of batches 3 and 4 compared to batch 2. P-values < 0.0001 are represented with ****, p-values < 0.001 are represented with ***, p-values < 0.01 are represented with ** and p-value <0.05 are represented with*.

TABLE 9

Challenge study conditions for each food tested

| | | | | | |
|---|---|---|---|---|---|
| Food tested | Details | Inoculation level (cfu/g) | Expiration date | 1.5 x expiration date | Storage conditions |
| 1 Pork tenderloins | Provigo | 10⁵ | 4 days | 7 days | 4° C., aerobically |
| 2 Atlantic salmon (filet) | Frandon Seafoods | 10³ | 4 days | 7 days | 4° C., aerobically |
| 3 Smoked salmon | Brand: Fumoir M. Émile Percé, Québec, Original recipe | 10³ | 14 days | 21 days | 4° C., vacuumed-packed |
| 4 RTE Chicken wieners, The original | MapleLodge Farms | 10³ | 2 days | 3 days | 4° C., aerobically |
| 5 Mushrooms | Carleton Mushrooms | 10^{1-1.5} | 5 days | 10 days | 7° C., aerobically |
| 6 Ham | duBreton | 10³ | 45 days | 68 days | 4° C. aerobically |
| 7 Milk and cream | CEFQ | 10³ | 1 day | 2 days | 7° C. aerobically |

TABLE 10

Enumeration of Listeria monocytogenes in diced pork tenderloin for each time of analysis

| Time | Enumeration of L. monocytogenes (cfu/g) | | |
|---|---|---|---|
| | CTL+ | CTL- | Pediocin PA-1 M31L (5) |
| Day 0 (4h) | 0 | 2337 ± 105 | 165 ± 60 |
| Day 1 (24h) | 0 | 2277 ± 91 | 90 ± 39 |
| Day 2 (48h) | 0 | 2402 ± 212 | 110 ± 71 |
| Day 4 | 0 | 2892 ± 120 | 35 ± 17 |
| Day 7 | 0 | 2892 ± 151 | 0 ± |

TABLE 11

Enumeration of Listeria monocytogenes in diced fresh salmon filet for each time of analysis

| Time | [0167] Enumeration of L. monocytogenes (cfu/g) | | |
|---|---|---|---|
| | CTL- | CTL+ | Pediocin PA-1 M31L (5) |
| Day 0 (4h) | 0 ± | 1081 ± 79 | 360 ± 167 |
| Day 3 | 0 ± 0 | 5755 ± 330 | 190 ± 62 |
| Day 7 | 0 ± 0 | 4744 ± 513 | 1021 ± 216 |
| Day 10 | 0 ± 0 | 31271 ± 15465 | 1431 ± 195 |

TABLE 12

Enumeration of Listeria monocytogenes in cold smoked salmon for each time of analysis

| Time | Enumeration of L. monocytogenes (cfu/g) | | | |
|---|---|---|---|---|
| | Batch 1 | Batch 2 | Batch 3 | Batch 4 |
| Day 0 (4h) | 0 | 300 ± 20 | 90 ± 30^{∗} | 155 ± 25 |
| Day 1 (24h) | 0 | 320 ± 0 | 110 ± 10^{∗} | 185 ± 75 |
| Day 2 (48h) | 0 | 305 ± 35 | 55 ± 5^{∗} | 105 ±35 |
| Day 7 | 0 | 260 ± 20 | 115 ± 35 | 180 ± 80 |
| Day 14 | 0 | 280 ± 40 | 55 ± 15^{∗} | 125 ± 5 |
| Day 21 | 0 | 691 ± 160 | 90 ± 30^{∗} | 90 ± 0^{∗} |

^{∗} Data means that the food reaches a level less than 100 cfu/g in ready-to-eat-food.

TABLE 13

Enumeration of Listeria monocytogenes in sliced ham for each time of analysis

| Time | Enumeration of L. monocytogenes (cfu/g) | | |
|---|---|---|---|
| | CTL - 1 | CTL + 2 | Pediocin PA-1 M31L (5) |
| Day 0 (4h) | 0 | 3179 ± 390 | 1631 ± 206 |
| Day 1 (24h) | 0 | 3129 ± 251 | 1247 ± 178 |
| Day 3 | 0 | 2932 ± 186 | 730 ± 73 |
| Day 7 | 0 | 2582 ± 475 | 480 ± 162 |
| Day 10 | 0 | 4277 ± 965 | 417 ± 112 |

TABLE 14

Enumeration of Listeria monocytogenes (cfu/ml and Log) in raw milk for each day of analysis

| Day | Group | Titre L. monocytogenes (cfu/ml) | Log (cfu/ml) | Log Reduction with pediocin PA-1 M31L (5) |
|---|---|---|---|---|
| 0 | Negative control | 0 | 0 | -0.11^{∗} |
| | Positive control | 1126 ± 198 | 3.04 ± 0.087 | |
| | Pediocin PA-1 M31L (5) | 865 ± 136 | 2.93 ± 0.072 | |
| 1 | Negative control | 0 | 0 | -0.79^{∗∗∗∗} |
| | Positive control | 1301 ± 285 | 3.10 ± 0.096 | |
| | Pediocin PA-1 M31L (5) | 225 ± 88 | 2.31 ± 0.207 | |
| 2 | Negative control | 0 | 0 | -1.50^{∗∗∗∗} |
| | Positive control | 1797 ± 127 | 3.25 ± 0.031 | |
| | Pediocin PA-1 M31L (5) | 70 ± 49 | 1.75 ± 0.312 | |

TABLE 15

Enumeration of Listeria monocytogenes (cfu/ml and Log) in raw cream for each day of analysis

| Day | Group | Titre L. monocytogenes (cfu/ml) | Log (cfu/ml) | Log Reduction with pediocin PA-1 M31L (5) |
|---|---|---|---|---|
| 0 | Negative control | 0 | 0 | -0.69^{∗∗∗∗} |
| | Positive control | 2177 ± 16 | 3.33 ± 0.003 | |
| | Pediocin PA-1 M31L | 550 ± 25 | 2.74 ± 0.019 | |
| 1 | Negative control | 0 | 0 | -1.10^{∗∗∗∗} |
| | Positive control | 1757 ± 223 | 3.24 ± 0.050 | |
| | Pediocin PA-1 M31L | 140 ± 25 | 2.14 ± 0.070 | |
| 2 | Negative control | 0 | 0 | -1.65^{∗∗∗∗} |
| | Positive control | 1972 ± 167 | 3.29 ± 0.038 | |
| | Pediocin PA-1 M31L | 50 ± 31 | 1.63 ± 0.24 | |

The results (cf. Tables 10-15) illustrate the antimicrobial activity of the food processing aid pediocin PA-1 M31L (5) as illustrated by the absence of Listeria spp. in the negative control (batch 1) of each food or a little presence of native microflora. Also, the positive control (batch 2) for each food shows a viable count of the inoculated strains of Listeria monocytogenes and this inoculum survives through the shelf life of the food. An immediate effect could be observed for pediocin PA-1 M31L (5) against the strains, as soon as day 0 (approximately 4 hours after the treatment). Also, in the majority of the matrices, treatment with pediocin PA-1 M31L (5) allows to reach less than 100 cfu/g of Listeria monocytogenes for ready-to-eat food (cf. salmon, smoked salmon, shrimp, poultry, and beef). For some food matrices tested, such as cheese curds and pork, a higher pathogen level was observed. However, these matrices were inoculated with a higher inoculum (10⁵ cfu/g). Interestingly, even if these matrices are inoculated with a higher inoculum, the reduction in Listeria is on average about 2 Log₁₀. The effect of pediocin PA-1 M31L (5) against Listeria monocytogenes is significant in every food matrix tested. Pediocin PA-1 M31L (5) advantageously exerts a rapid effect on Listeria monocytogenes while also bringing about only a very slow regrowth rate after its application.

Pediocin PA-1 M31L (5), when applied to a food matrix artificially contaminated with Listeria monocytogenes, is able to significantly reduce the amount of this pathogen on the food matrix. Batch 3, which received a treatment of 150 ng/g of pediocin PA-1 M31L (5), is always statistically different from the positive control (batch 2) with a p-value < 0.0001. The significant reduction of the amount of Listeria monocytogenes on the foods tested, for such small concentrations of pediocin PA-1 M31L (5), further illustrate its significant antimicrobial effect. Moreover, pediocin PA-1 M31L (5) has an immediate effect, since on day 0 (the day of the treatment), a reduction of Listeria monocytogenes could be observed. It is especially interesting to note that, despite the degradation of pediocin PA-1 M31L (5) on the food matrix following its application, its significant initial antimicrobial efficacy is still observed throughout the shelf life of the product.

### Example 14: Chicken Weiners

Samples were artificially inoculated with a cocktail of Listeria monocytogenes to reach a count of about 3Log per gram. The samples were subsequently divided into three groups. Firstly, a negative control to monitor if the product has naturally occurring Listeria; secondly, a positive control to measure the growth of Listeria monocytogenes during the experiments; and thirdly, samples having been treated with pediocin PA-1 M31L (5) to measure the control of the pathogen. The sample were analyzed on days 0, 1 and 5. With the data, the Log reduction was calculated for each day.

### Product Used: MapleLodge, The Original Chicken Wieners

Targeted microorganisms: Each group (except negative control) was inoculated with a cocktail composed of three strains of Listeria monocytogenes specific for RTE meat (cf. Table 8). The cocktail included a strain isolated from a listeriosis outbreak and three different serotypes (½a, ½b and 4b).

Group division: Each experiment was divided into three groups, every sample of 25 g ± 0.5 g was analysed in triplicate at each time point of the study. Group 1 is the negative control (the unmodified sausages); Group 2 is the positive control (the food inoculated with 1000 cfu/g of Listeria monocytogenes); and Group 3 was inoculated at the same level of group 2, but subsequently sprayed with pediocin PA-1 M31L (5) at 150 ng/g (150 ppb).

Food preparation: Sausages were weighed and placed in a clean plastic bag so that each sausage could be inoculated and sprayed.

Inoculation: Strains were grown aerobically in TSBYE at 37° C. for 24h and subcultured at 1% in the same conditions for 18h. Each strain of the group was then diluted to reach a contamination level of 1000 cfu/g of food. All the strains were pooled together to form the inoculation cocktail. All the samples of groups 2 and 3 were inoculated with 150 µl of the cocktail spread evenly on the sample. Pediocin PA-1 M31L (5) treatment: Samples were sprayed with a solution of pediocin PA-1 M31L (5) to reach a final dosage of 150 ppb, approximately 0.24 ml per sausage and 6.8 ml per kg. Selected results are illustrated in Table 16.

Storage conditions: Each sample was stored accordingly to the test conditions: 7° C. ± 0.5° C.

Sampling plan: Each sample was analysed at the day of the inoculation/treatment (2h, Day 0), at 24h (Day 1) and at day 5.

Enumeration method: MFLP-74 - Enumeration of Listeria monocytogenes in food, from the Compendium of Analytical Methods, Volume 3 of Health Canada. PALCAM - was used as the plating media.

Statistical analysis method: Figures were made using the JMP 10.0.0 program and statistical analysis were performed by comparing the batches together for each day separately with ANOVA and using the “Tukey-Kramer HSD” matrix to compare the means. P values on the graphs and tables display only the differences of group 2 compared to group 3. P values < 0.0001 are represented with ****, p values < 0.001 are represented with ***, p values < 0.01 are represented with ** and p values <0.05 are represented with *.

TABLE 16

Enumeration of Listeria monocytogenes in sausages for each experimental group at each analysis day

| Day | Sample group | Titre of L. monocytogenes (cfu/g) | Log (cfu/g) | Log reduction when using pediocin PA-1 M31L (5) |
|---|---|---|---|---|
| 0 | Negative control | 0 | 0 | -0.88^{∗∗∗∗} |
| | Positive control | 1691 ± 288 | 3.22 ± 0.07 | |
| | Pediocin PA-1 M31L (5) | 220 ± 45 | 2.33 ± 0.08 | |
| 1 | Negative control | 0 | 0 | -1.57^{∗∗∗∗} |
| | Positive control | 1811 ± 240 | 3.25 ± 0.05 | |
| | Pediocin PA-1 M31L (5) | 50 ± 17 | 1.67 ± 0.17 | |
| 5 | Negative control | 0 | 0 | -2.14^{∗∗∗∗} |
| | Positive control | 1721 ± 229 | 3.23 ± 0.05 | |
| | Pediocin PA-1 M31L (5) | 30 ± 30 | 1.08 ± 0.95 | |

The results (cf. Table 16) illustrate the antimicrobial activity of the food processing aid pediocin PA-1 M31L (5). To that effect, the use of pediocin PA-1 M31L (5) immediately reduces the amount of Listeria monocytogenes inoculated on the sausages by 0.88Log or 1471 cfu/g. Through the next several days, the antibacterial effect increases up to a reduction of 1.57 after 1 day and 2.14Log after 5 days. This illustrates that the sausages are compatible with pediocin PA-1 M31L (5) for a prolonged period of time. Moreover (cf. Table 16) after just 1 day, the level of L. monocytogenes decreases (and is stable up to 5 days) under 100 cfu/g, which is under the limit set for a category 2A RTE food by Health Canada (100 cfu/g), contrary to the positive control, which still has a high amount of the pathogen. Finally, the results illustrate that pediocin PA-1 M31L (5) is able to reduce and control the amount of Listeria monocytogenes present on RTE sausages. This shows the potential of this technology to provide safe products while using a clean label alternative.

### Example 15: Sliced Mushrooms

This example demonstrates the antimicrobial activity of pediocin PA-1 M31L (5) against the food-born pathogen Listeria monocytogenes in sliced mushrooms, and to monitor its growth during the shelf-life of the mushrooms.

Targeted microorganisms: Each group (except negative control) was inoculated with a cocktail composed of three strains of Listeria monocytogenes specific for vegetables (cf. Table 17). The cocktail included a strain isolated from a listeriosis outbreak and three different serotypes (½a, ½b, and 4b).

Group division: Each experiment was divided into three groups, every sample of 25 g ± 0.5 g was analyzed in triplicate at each time point of the study. Group 1 is the negative control (the unmodified mushrooms); Group 2 is the positive control (the food matrix inoculated with 10-30 cfu/g of Listeria monocytogenes); and Group 3 was inoculated at the same level as group 2 but subsequently sprayed with pediocin PA-1 M31L (5) at 150 ng/g (150 ppb).

Food preparation: Mushrooms were weighed and placed in a Petri dish so that each slice could be inoculated and sprayed.

Inoculation: Strains were grown aerobically in TSBYE at 37° C. for 24 h and subcultured at 1% in the same conditions for 18 h. Each strain of the group was then diluted to reach a contamination level of 10-30 cfu/g of food. All the strains are pooled together to form the inoculation cocktail. All the samples of groups 2 and 3 are inoculated with 500 µl of the cocktail spread evenly on the sample. Pediocin PA-1 M31L treatment: Group 3 was treated with pediocin PA-1 M31L (5) at the concentration of use. 1 ml ± 0.1 ml of the solution was sprayed onto the samples and left to dry before repackaging. Groups 1 and 2 were sprayed with distilled water to mimic the same humidity levels. Selected results are illustrated in Tables 17 and 18.

Storage conditions: Each sample was stored accordingly to the test conditions: 7° C. ± 0.5° C.

Sampling plan: Each sample was analyzed on the day of the inoculation/treatment (4h, Day 0), and on days 3, 5, 7, 10, and 12.

Frequency: The experiments were done in biological triplicates (n=3).

Enumeration method: MFLP-74 - Enumeration of Listeria monocytogenes in food, from the Compendium of Analytical Methods, Volume 3 of Health Canada. PALCAM - was used as the plating media.

Statistical analysis method: Figures were made with the JMP 10.0.0 program and statistical analysis were performed by comparing the batches together for each day separately with ANOVA and using the “Tukey-Kramer HSD” matrix to compare the means. P values on the graphs and tables display only the differences of group 2 compared to group 3. P values < 0.0001 are represented with ****, p values < 0.001 are represented with ***, p values < 0.01 are represented with ** and p value <0.05 are represented with *.

TABLE 17

Enumeration of Listeria monocytogenes in sliced mushrooms (in cfu/g and log) for each analysis day (average of n = 3)

| Group | Listeria monocytogenes count | | | | | |
|---|---|---|---|---|---|---|
| | Day 0 | | Day3 | | Day 5 | |
| | cfu/g | Log | cfu/g | Log | cfu/g | Log |
| 1 : Negative control | 0 | 0 | 0 | 0 | 0 | 0 |
| 2 : Positive control | 42 + 18 | 1.60 ± 0.16 | 21 ± 7 | 1.29 ± 0.21 | 26 ± 11 | 1.38 ± 0.18 |
| 3 : Pediocin PA-1 M31L | 9 ± 8 | 0.80 ± 0.45^{∗∗∗∗} | 9 ± 5 | 0.85 ± 0.38^{∗∗∗∗} | 23 ± 34 | 1.20 ± 0.57 |
| Group | Listeria monocytogenes count | | | | | |
| | Day 7 | | Day 10* | | Day 12* | |
| | cfu/g | Log | cfu/g | Log | cfu/g | Log |
| 1 : Negative control | 0 | 0 | 0 | 0 | 0 | 0 |
| 2 : Positive control | 28 ± 33 | 1.24 ± 0.43 | 20 ± 11 | 1.23 ± 0.29 | 650 ± 133 | 2.40 ± 0.83 |
| 3 : Pediocin PA-1 M31L | 4 ± 5 | 0.47 ± 0.41^{∗∗∗∗} | 9 ± 12 | 0.67 ± 0.55^{∗∗∗} | 27 ± 26 | 1.05 ± 0.72^{∗∗∗∗} |

TABLE 18

Growth of Listeria monocytogenes compared to day 0 and Log reduction when using pediocin PA-1 M31L (5) compared to the positive control (n=3)

| Group | Data in Log | | | | | |
|---|---|---|---|---|---|---|
| | Day 0 | | Day3 | | Day 5 | |
| | Reference data | Reduction when using pediocin PA-1 M31L | Growth since day 0 | Reduction when using pediocin PA-1 M31L | Growth since day 0 | Reduction when using pediocin PA-1 M31L |
| 1 : Negative control | 0 | 0 | 0 | 0 | 0 | 0 |
| 2 : Positive control | 1.60 | -0.8 | -0.3 | -0.44 | -0.22 | -1.24 |
| 3 : Pediocin PA-1 M31L | 0.80 | | +0.06 | | +0.41 | |
| Group | Data in Log | | | | | |
| | Day 7 | | Day 10 | | Day 12 | |
| | Growth since Day 0 | Reduction when using pediocin PA-1 M31L | Growth since day 0 | Reduction when using pediocin PA-1 M31L | Growth since day 0 | Reduction when using pediocin PA-1 M31L |
| 1 : Negative control | 0 | 0 | 0 | 0 | 0 | 0 |
| 2 : Positive control | -0.35 | -0.77 | -0.36 | -0.44 | +0.81 | -1.35 |
| 3 : Pediocin PA-1 M31L | -0.32 | | -0.12 | | +0.26 | |

The results (cf. Tables 17 and 18) illustrate the antimicrobial activity of the food processing aid pediocin PA-1 M31L (5). To that effect, when the mushrooms were sprayed with the recommended dosage of pediocin PA-1 M31L (5), there was an initial Log reduction of 0.80 compared to the positive control. The maximal Log reduction observed throughout the experiment was 1.35Log (on day 12), with an average reduction of 0.84Log for all days. Moreover, when using pediocin PA-1 M31L (5), the maximal growth observed was 0.41Log compared to 0.81Log for the untreated mushrooms. It is also important to consider that the normal microflora of mushrooms was usually very high from days 5 to 12, and that Listeria monocytogenes is not a competitive species. The fact that it fights for nutrients might explain that significant growth only occurs on day 12 and not before (in this particular case). Furthermore, the amount of L. monocytogenes, when using pediocin PA-1 M31L (5), is always significantly lower relative to the positive control with a p value < 0.0001 or 0.001, except on day 5, which may be attributed to the high standard deviation of the pediocin PA-1 M31L (5) group on day 5. It was determined that a good coverage method was essential to reach a maximum surface area of the mushrooms. Within laboratory conditions, this is the most variable parameter since the mushrooms are sprayed by hand. So, on the processing line, it will be very important to have a good spraying method, so that the growth of the pathogen may be well controlled. Finally, the efficacy of pediocin PA-1 M31L (5) can be observed as soon as 4 h (day 0) and has shown the ability to control the growth of Listeria monocytogenes for 12 days. When using pediocin PA-1 M31L (5), the maximal growth observed is 0.41Log and the maximal bacterial count is 27 cfu/g.

While the present disclosure has been described with reference to illustrative examples, it is to be understood that the disclosure is not limited to the disclosed examples. To the contrary, the disclosure is intended to cover various modifications and equivalent arrangements included within the spirit and scope of the appended claims.

### REFERENCES REFERRED TO IN THE SPECIFICATION

1. Cotter, P. D.; Ross, R. P.; Hill, C., Bacteriocins - a viable alternative to antibiotics? Nature reviews. Microbiology 2013, 11 (2), 95-105.

2. Abee, T.; Krockel, L.; Hill, C., Bacteriocins: modes of action and potentials in food preservation and control of food poisoning. International journal of food microbiology 1995, 28 (2), 169-85.

3. Bastos Mdo, C.; Coelho, M. L.; Santos, O. C., Resistance to bacteriocins produced by Gram-positive bacteria. Microbiology 2015, 161 (Pt 4), 683-700.

4. Duhan, J. S.; Nehra, K.; Gahlawat, S. K.; Saharan, P.; Surekha, D., Bacteriocins from Lactic Acid Bacteria. Biotechnology: Prospects and Applications, Salar, R. K.; Gahlawat, S. K.; Siwach, P.; Duhan, J. S., Eds. Springer India: New Delhi, 2013; pp 127-141.

5. Wolska, K. I.; Grzes, K.; Kurek, A., Synergy between novel antimicrobials and conventional antibiotics or bacteriocins. Pol J Microbiol 2012, 61 (2), 95-104.

6. Pattabiraman, V. R.; Bode, J. W., Rethinking amide bond synthesis. Nature 2011, 480 (7378), 471-9.

7. O’Bryan, C. A.; Koo, O. K.; Sostrin, M. L.; Ricke, S. C.; Crandall, P. G.; Johnson, M. G., Chapter 15 - Characteristics of Bacteriocins and Use as Food Antimicrobials in the United States. In Food and Feed Safety Systems and Analysis, Academic Press: 2018; pp273-286.

8. Rodríguez J.M., Martinez M.I., Kok J. 2002. Pediocin PA-1, a wide-spectrum bacteriocin from lactic acid bacteria. Crit. Rev. Food Sci. Nutr. 42:91-121.

9. Recalls, Market Withdrawals, & Safety Alerts | FDA.

10. Ostroff S. 2018. The Costs of Foodborne Illness, Product Recalls Make the Case for Food Safety Investments. Food Saf. Mag.

11. CDC. 2006. Estimated annual number of hospitalizations and deaths caused by 31 pathogens transmitted commonly by food Centers for Disease Control and Prevention.

12. Kuniyoshi T.M., O’Connor P.M., Lawton E, Thapa D, Mesa-Pereira B, Abulu S, Hill C, Ross R.P., Oliveira R.P.S., Cotter P.D. 2022. An oxidation resistant pediocin PA-1 derivative and penocin A display effective anti-Listeria activity in a model human gut environment. Gut Microbes 14:2004071.

13. Johnsen L, Fimland G, Eijsink V, Nissen-Meyer J. 2000. Engineering increased stability in the antimicrobial peptide pediocin PA-1. Appl. Environ. Microbiol 66:4798-4802.

14. Kuniyoshi T.M., Connor P.M.O., Arbulu S, Mesa-Pereira B, Oliveira R.P. de S, Hill C, Ross P, Cotter P.D. 2019. Expression, purification and antimicrobial activity of recombinant pediocin PA-1 M31L, a PA-1 derivative with enhanced stability. Access Microbiol. 1:739.

15. Bédard F, Hammami R, Zirah S, Rebuffat S, Fliss I, Biron E. 2018. Synthesis, antimicrobial activity and conformational analysis of the class IIa bacteriocin pediocin PA-1 and analogs thereof. Sci. Rep. 8:9029.

16. Klaenhammer T.R. 1993. Genetics of bacteriocins produced by lactic acid bacteria. FEMS Microbiol. Rev. 12:39-85.

17. Henderson J.T., Chopko A.L., van Wassenaar P.D.. 1992. Purification and primary structure of pediocin PA-1 produced by Pediococcus acidilactici PAC-1.0. Arch. Biochem. Biophys. 295:5-12.

18. Nieto Lozano J.C., Meyer J.N., Sletten K., Peláz C., Nes I.F. 1992. Purification and amino acid sequence of a bacteriocin produced by Pediococcus acidilactici. J. Gen. Microbiol. 138:1985-1990.

19. Wiggins G.L., Albritton W.L., Feeley J.C. 1978. Antibiotic susceptibility of clinical isolates of Listeria monocytogenes. Antimicrob. Agents Chemother. 13:854-860.

20. Mathur H., Field D., Rea M.C., Cotter P.D., Hill C., Ross R.P. 2017. Bacteriocin-Antimicrobial Synergy: A Medical and Food Perspective. Front. Microbiol. 8:1205.

21. Derksen, D. J.; Boudreau, M. A.; Vederas, J. C., Hydrophobic interactions as substitutes for a conserved disulfide linkage in the type IIa bacteriocins, leucocin A and pediocin PA-1. Chembiochem: a European journal of chemical biology 2008, 9 (12), 1898-901.

22. Oppegard, C.; Fimland, G.; Anonsen, J. H.; Nissen-Meyer, J., The Pediocin PA-1 Accessory Protein Ensures Correct Disulfide Bond Formation in the Antimicrobial Peptide Pediocin PA-1. Biochemistry 2015, 54 (19), 2967-2974.

23. Johnsen, L.; Fimland, G.; Eijsink, V.; Nissen-Meyer, J., Engineering increased stability in the antimicrobial peptide pediocin PA-1. Applied and environmental microbiology 2000, 66 (11), 4798-802.

24. Tiwari, S. K.; Sutyak Noll, K.; Cavera, V. L.; Chikindas, M. L., Improved antimicrobial activities of synthetic-hybrid bacteriocins designed from enterocin E50-52 and pediocin PA-1. Applied and environmental microbiology 2015, 81 (5), 1661-7.

25. O’Shea, E. F.; O’Connor, P. M.; O’Sullivan, O.; Cotter, P. D.; Ross, R. P.; Hill, C., Bactofencin A, a new type of cationic bacteriocin with unusual immunity. mBio 2013, 4 (6), e00498-13.

26. Kalchayanand N, Hanlin M.B., Ray B. 1992. Sublethal injury makes Gram-negative and resistant Gram-positive bacteria sensitive to the bacteriocins, pediocin AcH and nisin. Lett. Appl. Microbiol. 15:239-243.

27. Kalchayanand N, Sikes A, Dunne C.P., Ray B. 1998. Factors influencing death and injury of foodborne pathogens by hydrostatic pressure-pasteurization. Food Microbiol. 15:207-214.

28. National Advisory Committee on Microbiological criteria for Food. 2010. Parameters for determining inoculated pack/challenge study protocols. J. Food Prot. 73:140-202.

1. A method of preserving a food item comprising a food matrix, the method comprising applying a composition comprising a linear bacteriocin to a surface of the food item, wherein in situ disulfide bond formation occurs in the linear bacteriocin when the linear bacteriocin contacts the food matrix, and wherein the disulfide bond formation comprises oxidative coupling of a pair of thiol containing amino acid residues present in the linear bacteriocin.

2. The method of claim 1, wherein the linear bacteriocin is a gram-positive class II bacteriocin or a variant thereof.

3. The method of claim 2, wherein the gram-positive class II bacteriocin is a gram-positive class IIa bacteriocin or a variant thereof, a gram-positive class IIb bacteriocin or a variant thereof, a gram-positive class IIc bacteriocin or a variant thereof or a gram-positive class IId bacteriocin or a variant thereof.

4. The method of claim 3, wherein the gram-positive class IIa bacteriocin or variant thereof is a pediocin-like bacteriocin or variant thereof.

5. The method of claim 3, wherein the gram-positive class IId bacteriocin or variant thereof is a bactofencin-like bacteriocin or variant thereof.

6. The method of claim 2, wherein the variant has at least 80% sequence identity with an unmodified or native reference sequence.

7. The method of claim 6, wherein the variant has at least one of an amino acid substitution, modification, addition or deletion relative to an unmodified or native reference sequence.

8. The method of claim 1, wherein a solution comprising the linear bacteriocin is sprayed onto the surface of the food item.

9. The method of claim 8, wherein the solution comprises at least 50 ng/g of the linear bacteriocin.

10. The method of claim 1, wherein the food item is at least one of pork, salmon, ham, milk, cream, sausages or mushrooms.

11. The method of claim 10, wherein the pork is diced pork.

12. The method of claim 10, wherein the salmon is diced fresh salmon.

13. The method of claim 10, wherein the salmon is smoked salmon.

14. The method of claim 10, wherein the ham is sliced ham.

15. The method of claim 10, wherein the milk is raw milk.

16. The method of claim 10, wherein the cream is raw cream.

17. The method of claim 10, wherein the sausages are chicken wieners.

18. The method of claim 10, wherein the mushrooms are sliced mushrooms.

19. The method of claim 1, wherein the linear bacteriocin is effective for preserving a food item against Listeria monocytogenes, Clostridium perfringens, Clostridium botulinum, Salmonella typhimurium, Escherichia coli, Serratia liquefaciens or Pseudomonas fluorescens.

20. The method of claim 9, wherein the linear bacteriocin has the following sequence: KYYGNGVTCGKHSCSVDWGKATTCIINNGALAWATGGHQGNHKC.
